(19) United States
(12) Patent Application Publication
(10) Pub. No.: US 2005/0009777 A1
(43) Pub. Date: Jan. 13, 2005

(54) Non-natural carbon-linked nucleotides and dinucleotides

(76) Inventors: Stephen Robert Mack, Cambridge (GB); Verity Margaret Sabin, Cambridge (GB); Frances Celia Anne Galvin, Cambridge (GB)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/484,573

(22) PCT Filed: Jul. 25, 2002
(86) PCT No.: PCT/GB02/03417

(30) Foreign Application Priority Data
Jul. 25, 2001 (GB) 0118134.6
Mar. 14, 2002 (GB) 0205956.6

(51) Int. Cl. A61K 31/7076; A61K 31/7072; C07H 19/04

(52) U.S. Cl. 514/47; 514/50; 536/26.1

(57) ABSTRACT

Nucleotide derivatives of formula (1) are described, wherein: G is a hydrogen atom or an optionally substituted aliphatic, heteroaliphatic, cycloaliphatic, polycycloaliphatic, aromatic or heteroaromatic group or a non natural carbon-linked nucleoside as defined herein; G′ is a non-natural carbon-linked nucleoside as defined herein; n is zero, or the integer 1 or 2; m is zero or the integer 1 or 2; and the salts, solvates, hydrates and N-oxides thereof. The compounds are P2Y receptor agonists and are of use in the prophylaxis and treatment of diseases and disorders involving abnormal secretory mechanisms such as inadequate functioning of mucociliary clearance mechanisms or abnormal tear secretion or in the treatment of diseases involving inappropriate cellular glucose uptake.

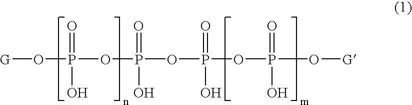

This invention relates to a series of non-natural carbon-linked nucleotides and dinucleotides, to compositions containing them, to processes for their preparation and to their use in medicine.

Extracellular nucleotides such as adenosine triphosphate (ATP), uridine triphosphate (UTP) and uridine diphosphate (UDP) play a fundamental role in mediating a number of physiological functions including, for example a general role in the control of secretions such as the clearance of retained mucus secretions and stimulation of ciliary beat frequency and particularly a central role in the coordination of mucociliary clearance mechanisms in the lung. Molecular biological techniques have allowed the discovery of several families of membrane bound receptors for these highly charged molecules. Thus, the P2X receptors are ligand-gated ion channels that are implicated in various neuromodulatory processes. The P2Y family of receptors are 7-transmembrane G-protein coupled receptors that bind both purine and pyrimidine nucleotides (Williams and Bhagwat, Ann. Rep. Med. Chem. 1996, 31, 21-30).

The P2Y receptors have been further subdivided into 9 subtypes known as P2Y1 to P2Y8 and P2Y11 (Fisher, B., Exp. Opin. Ther. Patents, 1999, 9, 385-399; Yerxa, B. R. and Johnson, F. L., Drugs of the Future, 1999, 24, 759-769). P2Y2 receptors have been implicated in the pathology of several disease states including lung diseases such as chronic obstructive pulmonary disease (which includes amongst others cystic fibrosis, chronic bronchitis and emphysema) and tear secretion, thrombosis, pain, cancer, sepsis and ischaemia-reperfusion injury.

P2Y2 receptors are found on the apical surface of airway epithelia and are believed to be the major coordinators of mucociliary clearance mechanisms in the lung. The continuous, cephalad movement of lower respiratory material is necessary for the clearance of inhaled pathogenic organisms or injurious particles and is essential to maintain airways necessary for efficient gas exchange. The movement of airway secretions, along with accompanying lumenal cells and free foreign particles is accomplished by the. actions of several cell types within the respiratory tract. Mucous is secreted by goblet cells and submucosal glands and forms a gel-like protective sheet within the lumen of the respiratory tract. The layer of mucus is propelled by the rhythmical, coordinated beat of the ciliated epithelial cells lining the airways from the terminal bronchi to the oropharynx and lining of the nose. The viscous mucous sheet would be immovable except that it floats on a much less viscous layer of fluid above the beating cilia. This periciliary fluid layer is maintained by the transport of ions (chloride and sodium) across the epithelium into the lumen of the airways followed by passive diffusion of water. Activation of P2Y2 by its presumed endogenous agonist, UTP, as well as by other nucleoside phosphates leads to increased mucociliary clearance presumably by increased chloride and water transport across the luminal surface (Mason, S. et al Br. J. Pharmacol., 1991, 103, 1649-1656), increased cilia beat frequency (Drutz, D. et al, Drug Dev. Res. 1996, 37, 185), increased mucin release (Lethem, M. et al, Am. J. Respir. Cell Mol. Biol., 1993, 9, 315-322) and increased surfactant release (Gobran, L. et al, Am. J. Physiol., 1994, 272, L187-196).

It has recently been discovered that the P2Y6 receptor, which selectively recognizes UDP as a potent ligand, also exists in airway tissue (International Patent Specification WO 99/09998). P2Y6 activation is also associated with chloride ion secretion and may play a role in coordination of mucociliary clearance mechanisms.

Cystic fibrosis (CF) is the most lethal genetic disease in Caucasians in the U.S.A., affecting approximately 1 in 2000 individuals (Fiel, S. B. et al, Semin. Respir. Crit. Care Med., 1994,15, 349-355), with median survival age being 30 years. CF occurs due to mutations in the gene that codes for the CF Transmembrane Conductance Regulator (CFTR) protein (Rommens, J. M. et al, Science, 1989, 245, 1059-1080). These mutations account for the abnormalities in sodium, chloride and water transport across epithelial cells resulting in dehydration and thickening of the mucus layer above the affected cells. The inability of CF patients to clear this thickened mucus and potential pathogens leads to chronic lung infection, progressive lung disease and impaired lung function, with lung infection accounting for 90% of deaths from CF.

New therapeutic approaches to the treatment of CF are required and one approach is the provision of agents that correct the underlying ion transport defects via physiological mechanisms that do not rely on the CFTR in order to normalize airway secretions, leading to improved mucociliary clearance and prevention of lung infections and damage. In this regard P2Y2 and P2Y6 receptor agonists may enhance mucociliary clearance by the mechanisms just mentioned. UTP, UDP and ATP have been demonstrated to activate chloride channel function, leading to hydration of lung mucin secretions (U.S. Pat. No. 5,292,498 and International Patent Specification WO99/09998) and increased ciliary beat frequency (Boucher, R. et al, Adenosine and Adenine Nucleotides: From Molecular Biology to Integrative Physiology, 525-532, Belardinelli, L. and Pelleg, A., Eds, Alumwer Academic Publishers, Boston, 1995) in the lung epithelial cells of CF patients. A combination of aerosolised UTP and amiloride has also been reported to improve mucociliary clearance from the peripheral airways of the lungs of CF patients to near normal levels (Bennett, W. D. et al, AM. J. Res. Critical Care Med. 1996, 153, 1796-1802).

Additionally a series of dinucleotides have also been disclosed as useful in the treatment of airway diseases including CF (U.S. Pat. No. 5,635,160), chronic bronchitis (International Patent Specifications WO 98/34942 and WO 99/61012) and sinusitis (U.S. Pat. No. 5,972,904).

Abnormal tear secretion can lead to dry eye disease, a general term for indications produced by abnormalities of the precorneal tear film characterised by a decrease in tear production or an increase in tear film evaporation, in combination with the resulting ocular surface disease. Current treatment of dry eye disease is limited to the use of artificial tears which is a short lived solution. Tear secretion may be stimulated from lacrimal accessory tissues via P2Y2 and/or P2Y4 purinergic receptor mediated mechanisms similar to those that hydrate airway epithelia, and agonists of these receptors may be useful in the treatment of dry eye disease.

P2Y2 and P2Y4 receptors also play a role in the control of glucose uptake into mammalian cardiac mycocytes (see International Patent Specification WO 99/43326). Use of agonists of these receptors to enhance glucose uptake may be used to minimize ischemic cardiac damage, such as that attributable to angina, myocardial infarction, cardiac arrhythmia, coronary artery disease, diabetes mellitus and cardiac ischemia attributable to shock, stress or exertion.

Since UTP, UDP and related dinucleotides are subject to rapid degradation in vivo their actions are likely to be short lived. Consequently there is a need for alternative long acting P2Y agonists. We have now found such a group of compounds which are potent longer acting P2Y agonists, particularly P2Y2, P2Y4 and/or P2Y6 agonists. The compounds are of use in medicine, for example in the treatment of diseases and disorders involving abnormal secretory mechanisms such as inadequate functioning of the mucociliary clearance mechanisms or abnormal tear secretion or in the treatment of diseases involving inappropriate cellular glucose uptake.

Thus according to one aspect of the invention we provide a compound of formula (1): 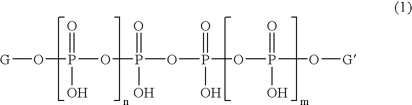
wherein:
G is a hydrogen atom or an optionally substituted aliphatic, heteroaliphatic, cycloaliphatic, heterocycloaliphatic, polycycloaliphatic, heteropolycycloaliphatic, aromatic or heteroaromatic group or a group of formula: 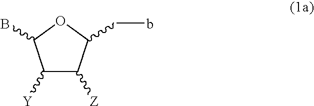
   in which Y and Z is each independently a hydrogen or halogen atom or a hydroxyl (—OH), alkoxy, azido (—N₃), amino (—NH₂), alkylamino or dialkylamino group, b represents the point of attachment to the remainder of the compound of formula (1) and B is an optionally substituted carbon-linked bicyclic heteroaromatic group:
G′ is a group of formula: 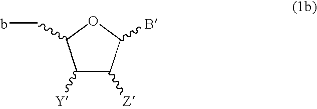
   in which B′ is an optionally substituted carbon-linked bicyclic heteroaromatic group, Z′ and Y′ is each an atom or group as previously defined for Z and b is as previously defined;
n is zero, or the integer 1 or 2;
m is zero or the integer 1 or 2;
and the salts, solvates, hydrates and N-oxides thereof for use in modulating P2Y receptor activity.

The compounds of formula (1) are potent agonists of P2Y receptors, particularly P2Y2, P2Y6 and/or P2Y4 receptors. The ability of the compounds to act in this way may be simply determined by employing tests such as those described hereinafter.

The compounds according to the invention are generally of use in modulating secretory processes and in particular are of use in the prophylaxis and treatment of lung diseases or disorders such as those involving inadequate functioning of the mucociliary clearance mechanisms such as chronic obstructive pulmonary disease and the invention extends to such a use and to the use of the compounds for the manufacture of a medicament for treating such diseases or disorders.

Diseases or disorders of this type include chronic bronchitis, Primary Ciliary Dyskinesia and cystic fibrosis. Additionally compounds according to the invention may be used in the prevention of pneumonia due to immobility. Furthermore, due to their general ability to increase hydration, clear retained mucus secretions and stimulate ciliary beat frequency, the compounds according to the invention are also useful in the treatment of sinusitis, otitis media, post-operative mucous retention, nasolacrimal duct obstructions, female infertility or irritation caused by vaginal dryness and nasolacrimal duct obstructions. In addition the compounds according to the present invention are useful for treating dry eye and retinal detachment. The compounds may also be of use in the control of glucose uptake in mammalian cardiac mycocytes.

For the prophylaxis or treatment of disease the compounds according to the invention may be administered as pharmaceutical compositions, and according to a further aspect of the invention we provide a pharmaceutical composition which comprises a compound of formula (1) together with one or more pharmaceutically acceptable carriers, excipients or diluents.

Pharmaceutical compositions according to the invention may take a form suitable for oral, buccal, parenteral, nasal, topical, vaginal or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles and preservatives. The preparations may also contain buffer salts, flavouring, colouring and sweetening agents as appropriate.

Preparations for oral administration may be suitably formulated to give controlled release of the active compound.

For buccal administration the compositions may take the form of tablets or lozenges formulated in conventional manner.

The compounds for formula (1) may be formulated for parenteral administration by injection e.g. by bolus injection or infusion. Formulations for injection may be presented in unit dosage form, e.g. in glass ampoule or multi dose containers, e.g. glass vials. The compositions for injection may take such forms as suspensions, solutions. or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use. For particle mediated administration the compounds of formula (1) may be coated on particles such as microscopic gold particles.

In addition to the formulations described above, the compounds of formula (1) may also be formulated as a depot preparation. Such long acting formulations may be administered by implantation or by intramuscular injection.

For nasal administration or administration by inhalation, the compounds for use according to the present invention are conveniently delivered in the form of an aerosol spray presentation for pressurised packs or a nebuliser, with the use of suitable propellant, e.g. dichlorodifluoromethane, trichlorofluoromethane, dichlorotetrafluoroethane, carbon dioxide or other suitable gas or mixture of gases.

For topical administration the compounds of formula (1) may be delivered in the form of a liquid or gel suspension in the form of drops, spray or gel. These formulations may be prepared by mixing the active ingredient with a suitable physiologically compatible vehicle. Such vehicles include for example saline solution, water soluble polyethers such as polyethylene glycol, polyvinyls such as polyvinyl alcohol, cellulose derivatives such as methylcellulose, petroleum derivatives such as mineral oil and white petroleum, animal fats such as lanolin, polymers of acrylic acid such as carboxypolymethylene gel, vegetable fats such as peanut oil and polysaccharides such as dextrans.

For vaginal or rectal administration the compounds of formula (1) may be formulated as a suppository. These formulations may be prepared by mixing the active ingredient with a suitable non-irritating excipient which is a solid at room temperature but liquid at the body temperature. Such materials include for example cocoa butter and polyethylene glycols.

The compositions may, if desired, be presented in a pack or dispenser device which may contain one or more unit dosage forms containing the active ingredient. The pack or dispensing device may be accompanied by instructions for administration.

The quantity of a compound of the invention required for the prophylaxis or treatment of a particular condition will vary depending on the compound chosen, and the condition of the patient to be treated. In general, however, daily dosages may range from around 100 ng/kg to 100 mg/kg e.g. around 0.01 mg/kg to 40mg/kg body weight for oral or buccal administration, from around 10 ng/kg to 50 mg/kg body weight for parenteral administration and around 0.05 mg to around 1000 mg e.g. around 0.5 mg to around 1000 mg for nasal administration or administration by inhalation or insufflation.

Particular compounds of formula (1) form a further aspect of the invention and in a further aspect we therefore provide a compound of formula (1c): 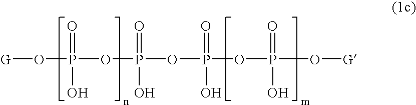
wherein:
G is a hydrogen atom or an optionally substituted aliphatic, heteroaliphatic, cycloaliphatic, heterocycloaliphatic, polycycloaliphatic, heteropolycycloaliphatic, aromatic or heteroaromatic group or a group of formula: 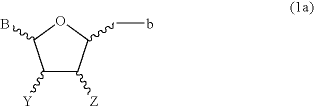
   in which Y and Z is each independently a hydrogen or halogen atom or a hydroxyl (—OH), alkoxy, azido (—N₃), amino (—NH₂), alkylamino or dialkylamino group, b represents the point of attachment to the remainder of the compound of formula (1) and B is an optionally substituted carbon-linked bicyclic heteroaromatic group;
G′ is a group of formula: 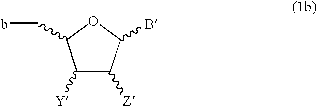
   in which B′ is an optionally substituted carbon-linked bicyclic heteroaromatic group, Z′ and Y′ is each an atom or group as previously defined for Z and b is as previously defined;
n is zero, or the integer 1 or 2;
m is zero or the integer 1 or 2;
   provided that:
   1) when n and m is each zero, G is a hydrogen atom and G′ is a group of formula (1b) in which Y′ and Z′ is each a hydroxyl (—OH) group then B′ is other than a 7H-pyrazolo[4,3-d]pyrimidine-7-one-3-yl, 7-amino-1H-pyrazolo[4,3-d]pyrimidin-3-yl, 4H-pyrrolo[3,2-d]pyrimidin-4-one-7-yl or 4-amino-4H-pyrrolo[3,2-d]pyrimidin-7-yl group;
   2) when one of n and m is the integer 1 and the other is zero, G is a hydrogen atom and G′ is a group of formula (1b) in which Y′ and Z′ is each a hydroxyl (—OH) group then B′ is other than a 7H-pyrazolo[4,3-d]pyrimidine-7-one-3-yl, 5-amino-7H-pyrazolo[4,3-d]pyrimidine-7-one-3-yl, 7-amino-1H-pyrazolo[4,3-d]pyrimidin-3-yl, 2-amino-4H-pyrrolo[3,2-d]pyrimidin-4-one-7-yl, 4H-pyrrolo[3,2-d]pyrimidin-4-one-7-yl, 2-amino-4H-pyrrolo[3,2-d]pyrimidin-4-one-7-yl, 4-aminothieno[3,2-d]pyrimidin-7-yl, 4-amino-4H-pyrrolo[3,2-d]pyrimidin-7-yl or 4-amino-5H-pyrrolo[3,2-d]pyrimidin-7-yl group;
   3) when n and m is each zero, G′ is a group of formula (1b) in which Y, and Z′ is each a hydroxyl (—OH) group and B′ is a 7-amino-1H-pyrazolo[4,3-d]pyrimidin-3-yl group and G is a group of formula (1a) in which Y′ and Z′ is each a hydroxyl (—OH) group then B is other than a 7-amino-1H-pyrazolo[4,3-d]pyrimidin-3-yl group;
      and the salts, solvates, hydrates and N-oxides thereof.

It will be appreciated that compound of formulae (1), (1a), (1b), and (1c) may have one or more chiral centres, and exist as enantiomers or diastereomers (for example as indicated by wiggly lines in formula (1a)). The invention is understood to extend to all such enantiomers, diastereomers and mixtures thereof, including racemates. In addition, compounds of formula (1), (1a), (1b) and (1c) may exist as tautomers, for example keto (CH₂C═O)-enol (CH═CHOH) tautomers. Formulae (1), (1a), (1b) and (1c) and the formulae hereinafter are intended to represent all individual isomers, tautomers and mixtures thereof unless stated or shown otherwise.

The following general terms as used herein have the stated meaning unless specifically described otherwise.

As used herein the term “alkyl” whether present as a group or part of a group includes straight or branched C₁₋₆alkyl groups, for example C₁₋₄alkyl groups such as methyl, ethyl, n-propyl, i-propyl or t-butyl groups. Similarly, the terms “alkenyl” or “alkynyl” are intended to mean straight or branched C₂₋₆alkenyl or C₂₋₆alkynyl groups such as C₂₋₄alkenyl or C₂₋₄alkynyl groups. Optional substituents which may be present on these groups include those optional substituents mentioned hereinafter in relation to G when G is an optionally substituted aliphatic group.

The term halogen is intended to include fluorine, chlorine, bromine or iodine atoms.

The term “haloalkyl” is intended to include those alkyl groups just mentioned substituted by one, two or three of the halogen atoms just described. Particular examples of such groups include —CF₃, —CCl₃, —CHF₂, —CHCl₂, —CH₂F and —CH₂Cl groups.

The term “alkoxy” as used herein is intended to include straight or branched C₁₋₆alkoxy e.g. C₁₋₄alkoxy such as methoxy, ethoxy, n-propoxy, i-propoxy and t-butoxy. “Haloalkoxy” as used herein includes any of these alkoxy groups substituted by one, two or three halogen atoms as described above. Particular examples include —OCF₃, —OCCl₃, —OCHF₂, —OCHCl₂, —OCH₂F and —OCH₂Cl groups.

As used herein the term “alkylthio” is intended to include straight or branched C₁₋₆alkylthio, e.g. C₁₋₄alkylthio such as methylthio or ethylthio.

As used herein the term “alkylamino or dialkylamino” is intended to include the groups —NHR¹ and —N(R¹)₂ [where R¹ is an optionally substituted straight or branched alkyl group]. Where two R¹ groups are present these may be the same or different. In addition where two R¹ groups are present these may be joined together with the N atom to which they are attached to form an optionally substituted heterocycloalkyl group which may contain a further heteroatom or heteroatom containing group such as an —O— or —S— atom or —N(R¹)— group. Particular examples of such optionally substituted heterocycloalkyl groups include optionally substituted pyrrolidinyl, piperidinyl, morpholinyl, thiomorpholinyl and N′-C₁₋₆alkyl-piperazinyl groups. The optional substituents which may be present on such heterocycloalkyl groups include those optional substituents as described hereinafter in relation to aliphatic groups.

When the group G is present in compounds of formula (1) as an optionally substituted aliphatic group it may be an optionally substituted C₁₋₁₀aliphatic chain. Particular examples include optionally substituted straight or branched chain C₁₋₆alkylene, C₂₋₆alkenylene, or C₂₋₆alkynylene groups.

Particular examples of aliphatic groups represented by G include optionally substituted —CH₃, —CH₂CH₃, —CH(CH₃)CH₃, —(CH₂)₂CH₃, —(CH₂)₃CH₃, —CH(CH₃)(CH₂)₂CH₃, —CH₂CH(CH₃)CH₃, —C(CH₃)₂CH₃, —CH₂C(CH₃)₂CH₃, —(CH₂)₂CH(CH₃)CH₃, —CH(CH₃)CH₂CH₃, —CH(CH₃)CH₂CH(CH₃)₂, —CH₂CH (CH₃)CH₂CH₃, —(CH₂)₂C(CH₃)₃, —(CH₂)₄CH₃, —(CH₂)₅CH₃, —CHCH₂, —CHCHCH₃, —CH₂CHCH₂, —CHCHCH₂CH₃, —CH₂CHCHCH₃, —(CH₂)₂CHCH₂, —CCH, —CCCH₃, —CH₂CCH, —CCCH₂CH₃, —CH₂CCCH₃ or —(CH₂)₂CCH₂ groups.

Heteroaliphatic groups represented by the group G in the compounds of formula (1) include the aliphatic groups just described but with each additionally containing one, two, three or four heteroatoms or heteroatom-containing groups. Particular heteroatoms or groups include atoms or groups L¹ where L¹ is a linker atom or group. Each L¹ atom or group may interrupt the aliphatic group, or may be positioned at its terminal carbon atom to connect the group to an adjoining atom or group. Particular examples include optionally substituted -L¹CH₃, —CH₂L¹CH₃, -L¹CH₂CH₃, —CH₂L¹CH₂CH₃, —(CH₂)₂L¹CH₃, —(CH₂)₃L¹CH₃, -L¹(CH₂)₃, -L¹CH₂CHCH₂ and —(CH₂)₂L¹CH₂CH₃ groups.

When L¹ is present in heteroaliphatic groups as a linker atom or group it may be any divalent linking atom or group. Particular examples include —O— or —S— atoms or —C(O)—, —C(O)O—, —OC(O)—, —C(S)—, —S(O)—, —S(O)₂—, —N(R²)— [where R² is a hydrogen atom or a straight or branched alkyl group], —N(R²)O—, —N(R²)N—, —CON(R²)—, —OC(O)N(R²)—, —CSN(R²)—, —N(R²)CO—, —N(R²)C(O)O—, —N(R²)CS—, —S(O)₂N(R²)—, —N(R²)S(O)₂—, —N(R²)CON(R²)—, —N(R²)CSN(R²)— or —N(R²)SO₂N(R²)— groups. Where L¹ contains two R² groups these may be the same or different.

The optional substituents which may be present on aliphatic or heteroaliphatic groups represented by G include one, two, three or more substituents where each substituent may be the same or different and is selected from halogen atoms, e.g. fluorine, chlorine, bromine or iodine atoms, or —OH, —CO₂H, —CO₂R³ [where R³ is an optionally substituted straight or branched C₁₋₆alkyl group], e.g. —CO₂CH₃ or —CO₂C(CH₃)₃, —CONHR³, e.g. —CONHCH₃, —CON(R³)₂, e.g. —CON(CH₃)₂, —COR³, e.g. —COCH₃, C₁₋₆alkoxy, e.g. methoxy or ethoxy, haloC₁₋₆alkoxy, e.g. trifluoromethoxy or difluoromethoxy, thiol (—SH) —S(O)R³, e.g. —S(O)CH₃, —S(O)₂R³, e.g. —S(O)₂CH₃, C₁₋₆alkylthio e.g. methylthio or ethylthio, amino, alkylamino, e.g. —NHCH₃, dialkylamino, e.g. —N(CH₃)₂ or optionally substituted cycloaliphatic, heterocycloaliphatic, aromatic or heteroaromatic groups. Where two R³ groups are present in any of the above substituents these may be the same or different. Optionally substituted cycloaliphatic and heterocycloaliphatic groups include those optionally substituted cycloaliphatic and heterocycloaliphatic groups and optional substitutuents described hereinafter in relation to G, especially optionally substituted cyclopropyl, cyclobutyl, cyclopentyl, cyclohexyl, cyclopentenyl, tetrahydrofuranyl, dihydrofuranyl and tetrahydropyranyl groups. Optionally substituted aromatic and heteroaromatic groups include those optionally substituted aromatic and heteroaromatic groups and optional substituents described hereinafter in relation to G, especially optionally substituted phenyl, thienyl, furanyl, pyridyl and pyrimidinyl groups.

Optionally substituted cycloaliphatic groups represented by the group G in compounds of the invention include optionally substituted C₃₋₁₀cycloaliphatic groups. Particular examples include optionally substituted C₃₋₁₀cycloalkyl, e.g. C₃₋₇cycloalkyl or C₃₋₁₀cycloalkenyl, e.g. C₃₋₇cycloalkenyl groups.

Optionally substituted heterocycloaliphatic group represented by the group G include optionally substituted C₃₋₁₀heterocycloaliphatic group. Particular examples include optionally substituted C₃₋₁₀heterocycloalkyl, e.g. C₃₋₇heterocycloalkyl or C₃₋₁₀heterocycloalkenyl, e.g. C₃₋₇heterocycloalkenyl groups, each of said groups containing one, two, three or four heteroatoms or heteroatom containing groups L² where L² is an atom or group as previously defined for L¹.

Optionally substituted polycycloaliphatic groups represented by the group G include optionally substituted C₇₋₁₀bi- or tricycloalkyl or C₇₋₁₀bi- or tricycloalkenyl groups. Optionally substituted heteropolycycloaliphatic groups represented by the group G include optionally substituted C₇₋₁₀bi- or tricycloalkyl or C₇₋₁₀bi- or tri-cycloalkenyl groups containing one, two, three, four or more L² atoms or groups.

Particular examples of cycloaliphatic, polycycloaliphatic, heterocycloaliphatic and heteropolycycloaliphatic groups represented by the group G include optionally substituted cyclopropyl, cyclobutyl, cyclopentyl, cyclohexyl, cycloheptyl, 2-cyclobuten-1-yl, 2-cyclopenten-1-yl, 3-cyclopenten-1-yl, adamantyl, norbornyl, norbornenyl, dihydrofuranyl, tetrahydrofuranyl, tetrahydropyranyl, dihydrothiophenyl, tetrahydrothiophenyl, pyrroline, e.g. 2- or 3-pyrrolinyl, pyrrolidinyl, pyrrolidinone, oxazolidinyl, oxazolidinone, dioxolanyl, e.g. 1,3-dioxolanyl, imidazolinyl, e.g. 2-imidazolinyl, imidazolidinyl, pyrazolinyl, e.g. 2-pyrazolinyl, pyrazolidinyl, 5,6-dihydro-2(1H)-pyrazinone, tetrahydropyrimidinyl, thiazolinyl, thiazolidinyl, pyranyl, e.g. 2- or 4-pyranyl, piperidinyl, homopiperidinyl, heptamethyleneiminyl, piperidinone, 1,4-dioxanyl, morpholinyl, morpholinone, 1,4-dithianyl, thiomorpholinyl, piperazinyl, homopiperazinyl, 1,3,5-trithianyl, oxazinyl, e.g. 2H-1,3-, 6H-1,3-, 6H-1,2-, 2H-1,2- or 4H-1,4-oxazinyl, 1,2,5-oxathiazinyl, isoxazinyl, e.g. o- or p-isoxazinyl, oxathiazinyl, e.g. 1,2,5 or 1,2,6-oxathiazinyl, 1,3,5-oxadiazinyl, dihydroisothiazolyl, dihydroisothiazole 1,1-dioxide , e.g. 2,3-dihydroisothiazole 1,1-dioxide, dihydropyrazinyl and tetrahydropyrazinyl groups.

The optional substituents which may be present on the cycloaliphatic, polycycloaliphatic, heterocycloaliphatic or polyheterocycloaliphatic groups represented by the group G include one, two, three or more substituents selected from halogen atoms, or C₁₋₆alkyl, e.g. methyl or ethyl, haloC₁₋₆alkyl, e.g. halomethyl or haloethyl such as difluoromethyl or trifluoromethyl, optionally substituted by hydroxyl, e.g. —C(OH)(CF₃)₂, C₁₋₆alkoxy, e.g. methoxy or ethoxy, haloC₁₋₆alkoxy, e.g. halomethoxy or haloethoxy such as difluoromethoxy or trifluoromethoxy, thiol, C₁₋₆alkylthiol, e.g. methylthiol or ethylthiol, carbonyl (═O), thiocarbonyl (═S), imino (═NR^{4a}) [where R^{4a} is an —OH group or a C₁₋₆alkyl group], or -(Alk¹)ᵥR⁵ groups in which Alk¹ is a straight or branched C₁₋₃alkylene chain, v is zero or the integer 1 and R⁵ is a C₅₋₈cycloalkyl, —OH, —SH, —N(R⁶)(R⁷) [in which R⁶ and R⁷ is each independently selected from a hydrogen atom or an optionally substituted alkyl or C₃₋₈cycloalkyl group], —OR⁶, —SR⁶, —CN, —NO₂, —CO₂R⁶, —SOR⁶, —SO₂R⁶, —SO₃R⁶, —OCO₂R⁶, —C(O)R⁶, —OC(O)R⁶, —C(S)R⁶, —C(O)N(R⁶) (R⁷), —OC(O)N(R⁶)(R⁷), —N(R⁶)C(O)R⁷, —C(S) N(R⁶)(R⁷), —N(R⁶)C(S)R⁷, —SO₂N(R⁶)(R⁷), —N(R⁶)SO₂R⁷, —N(R⁶)C(O)N(R⁷)(R⁸) [where R⁸ is as defined for R⁶], —N(R⁶)C(S)N(R⁷)(R⁸), —N(R⁶)SO₂N(R⁷)(R⁸) or an optionally substituted aromatic or heteroaromatic group.

Particular examples of Alk¹ chains include —CH₂—, —CH₂CH₂—, —CH₂CH₂CH₂— and —CH(CH₃)CH₂— chains.

When R⁵, R⁶, R⁷ and/or R⁸ is present as a C₃₋₈cycloalkyl groups it may be for example a cyclopropyl, cyclobutyl, cyclopentyl or cyclohexyl group. Optional substituents which may be present on such groups include for example one, two or three substituents which may be the same or different selected from halogen atoms, for example fluorine, chlorine, bromine or iodine atoms, or hydroxy or C₁₋₆alkoxy, e.g. methoxy, ethoxy or i-propoxy groups.

When the groups R⁶ and R⁷ or R⁷ and R⁸ are both alkyl groups these groups may be joined, together with the N atom to which they are attached, to form a heterocyclic ring. Such heterocyclic rings may be optionally interrupted by a further heteroatom or heteroatom containing group selected from —O—, —S—, —N(R⁷)—, —C(O)— or —C(S)— groups. Particular examples of such heterocyclic rings include piperidinyl, pyrazolidinyl, morpholinyl, thiomorpholinyl, pyrrolidinyl, imidazolidinyl and piperazinyl rings.

Additionally, when the group G is a heterocycloaliphatic or polyheterocycloaliphatic group containing one or more nitrogen atoms each nitrogen atom may be optionally substituted by a group -(L³)ₚ(Alk²)_{q}R⁹ in which L³ is a —C(O)—, —C(O)O—, —C(S)—, —S(O)₂—, —CON(R⁶)— or —SO₂N(R⁶)—; p is zero or the integer 1; Alk² is an optionally substituted aliphatic or heteoaliphatic chain; q is zero or the integer 1; and R⁹ is a hydrogen atom or an optionally substituted cycloaliphatic, heterocycloaliphatic, polycycloaliphatic, heteropolycycloaliphatic, aromatic or heteroaromatic group.

When Alk² is present as an aliphatic or heteroaliphatic chain it may be for example any divalent chain corresponding to the above-mentioned aliphatic or heteroaliphatic groups described for G, where a terminal hydrogen atom is replaced by a bond.

Aromatic or heteroaromatic groups represented by R⁵ and/or R⁹ include those aromatic and heteroaromatic groups as mentioned hereinafter in relation to the group G. Optional substituents on these groups include those substituents as described hereinafter for those aromatic and heteroaromatic groups represented by G.

Cycloaliphatic, heterocycloaliphatic, polycycloaliphatic or heteropolycycloaliphatic groups represented by R⁹ include those groups as described hereinbefore for the group G. Optional substituents which may be present on these groups include those described in relation to G when G is a cycloaliphatic, heterocycloaliphatic, polycycloaliphatic or heteropolycycloaliphatic group.

Optionally substituted aromatic groups represented by the group G include for example monocyclic or bicyclic fused ring C₆₋₁₂aromatic groups, such as phenyl, 1- or 2-napthyl, 1- or 2-tetrahydronapthyl, indanyl or indenyl groups.

Heteroaromatic groups represented by the group G include for example C₁₋₉heteroaromatic groups containing for example one, two, three or four heteroatoms selected from oxygen, sulphur or nitrogen atoms. In general, the heteroaromatic groups may be for example monocyclic or bicyclic fused ring heteroaromatic groups. Monocyclic heteroaromatic groups include for example five- or six-membered heteroaromatic groups containing one, two, three or four heteroatoms selected from oxygen, sulphur or nitrogen atoms. Bicyclic heteroaromatic groups include for example eight- to thirteen-membered fused ring heteroaromatic groups containing one, two or more heteroatoms selected from oxygen, sulphur or nitrogen atoms.

Particular examples of heteroaromatic groups of these types include pyrrolyl, furyl, imidazolyl, N—C₁₋₆alkylimidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,5-oxadiazolyl, 1,3,4-oxadiazolyl, 1,3,4-thiadiazolyl, pyridyl, pyrimidinyl, pyridazinyl, pyrazinyl, 1,3,5-triazinyl, 1,2,4-triazinyl, 1,2,3-triazinyl, benzofuryl, [2,3-dihydro]benzofuryl, benzothienyl, [2,3-dihydro]benzothienyl, benzotriazolyl, indolyl, indolinyl, indazolinyl, benzimidazolyl, imidazo[1,2-a]pyridyl, benzothiazolyl, benzoxazolyl, benzisoxazolyl, benzopyranyl, [3,4-dihydro]benzopyranyl, quinazolinyl, quinoxalinyl, naphthyridinyl, imidazo[1,5-a]pyridinyl, imidazo[1,5-a]pyrazinyl, imidazo[1,5-c]pyrimidinyl, pyrido[3,4-b]pyridyl, pyrido[3,2-b]pyridyl, pyrido[4,3-b]pyridyl, quinolinyl, isoquinolinyl, phthalazinyl, tetrazolyl, 5,6,7,8-tetrahydroquinolinyl, 5,6,7,8-tetrahydroisoquinolinyl, imidyl, e.g. succinimidyl, phthalimidyl or naphthalimidyl such as 1,8-naphthalimidyl, pyrazolo[4,3-d]pyrimidinyl, furo[3,2-d]pyrimidinyl, thieno[3,2-d]pyrimidinyl, pyrrolo[3,2-d]pyrimidinyl, pyrazolo[3,2-b]pyridinyl, furo[3,2-b]pyridinyl, thieno[3,2-b]pyridinyl, pyrrolo[3,2-b]pyridinyl, thiazolo[3,2-a]pyridinyl, pyrido[1,2-a]pyrimidinyl, tetrahydroimidazo[1,2-a]pyrimidinyl and dihydroimidazo[1,2-a]pyrimidinyl groups.

Optional substituents which may be present on aromatic or heteroaromatic groups represented by the group G include one, two, three or more substituents, each selected from an atom or group R¹⁰ in which R¹⁰ is R^{10a} or -Alk³(R^{10a})ᵣ, where R^{10a} is a halogen atom, or an amino (—NH₂), substituted amino, nitro, cyano, amidino, hydroxyl (—OH), substituted hydroxyl, formyl, carboxyl (—CO₂H), esterified carboxyl, thiol (—SH), substituted thiol, —COR¹¹ [where R¹¹ is an -Alk³(R^{10a})ᵣ, aryl or heteroaryl group], —CSR¹¹, —SO₃H, —SOR¹¹, —SO₂R¹¹, —SO₃R¹¹, —SO₂NH₂, —SO₂NHR¹¹, —SO₂N(R¹¹)₂, —CONH₂, —CSNH₂, —CONHR¹¹, —CSNHR¹¹, —CON(R¹¹)₂, —CSN(R¹¹)₂, —N(R¹²)SO₂R¹¹ [where R¹² is a hydrogen atom or a straight or branched alkyl group], —N(SO₂R¹¹)₂, —N(R¹²)SO₂NH₂, —N(R¹²)SO₂NHR¹¹, —N(R¹²)SO₂N(R¹¹)₂, —N(R¹²)COR¹¹, —N(R¹²)CONH₂, —N(R¹²)CONHR¹¹, —N(R¹²)CON(R¹¹)₂, —N(R¹²)CSNH₂, —N(R¹²)CSNHR¹¹, —N(R¹²)CSN(R¹¹)₂, —N(R¹²)CSR¹¹, —N(R¹²)C(O)OR¹¹, —SO₂NHet¹ [where —NHet¹ is an optionally substituted C₅₋₇cyclicamino group optionally containing one or more other —O— or —S— atoms or —N(R¹²)—, —C(O)— or —C(S)— groups], —CONHet¹, —CSNHet¹, —N(R¹²)SO₂NHet¹, —N(R¹²)CONHet¹, —N(R¹²)CSNHet¹, —SO₂N(R¹²)Het [where -Het is as previously defined], -Het, —CON(R¹²)Het, —CSN(R¹²)Het, —N(R¹²)CON(R¹²)Het, —N(R¹²)CSN(R¹²)Het, —N(R¹²)SO₂N(R¹²)Het, aryl or heteroaryl groups; Alk³ is a straight or branched C₁₋₆alkylene, C₂₋₆alkenylene or C₂₋₆alkynylene chain, optionally interrupted by one, two or three —O— or —S— atoms or —S(O)ₙ— [where n is an integer 1 or 2] or —N(R¹²)— e.g. —N(CH₃)— groups; and r is zero or the integer 1, 2, or 3. It will be appreciated that when two R¹¹ or R¹² groups are present in one of the above substituents the R¹¹ and R¹² groups may be the same or different.

When in the group -Alk³(R^{10a})ₘ r is an integer 1, 2 or 3, it is to be understood that the substituent or substituents R^{10a} may be present on any suitable carbon atom in -Alk³. Where more than one R^{10a} substituent is present these may be the same or different and may be present on the same or different atom in -Alk³. Clearly, when r is zero and no substituent R^{10a} is present the alkylene, alkenylene or alkynylene chain represented by Alk³ becomes an alkyl, alkenyl or alkynyl group.

When R^{10a} is a substituted amino group it may be for example a group —NHR¹¹ [where R¹¹ is as defined above] or a group —N(R¹¹)₂ wherein each R¹¹ group is the same or different.

When R^{10a} is a halogen atom it may be for example a fluorine, chlorine, bromine, or iodine atom.

When R^{10a} is a substituted hydroxyl or substituted thiol group it may be for example a group —OR¹¹ or a —SR¹² or —SC(═NH)NH₂group respectively.

Esterified carboxyl groups represented by the group R^{10a} include groups of formula —CO₂Alk⁴ wherein Alk⁴ is a straight or branched, optionally substituted C₁₋₈alkyl group such as a methyl, ethyl, n-propyl, i-propyl, n-butyl, i-butyl, s-butyl or t-butyl group; a C₆₋₁₂arylC₁₋₈alkyl group such as an optionally substituted benzyl, phenylethyl, phenylpropyl, 1-naphthylmethyl or 2-naphthylmethyl group; a C₆₋₁₂aryl group such as an optionally substituted phenyl, 1-naphthyl or 2-naphthyl group; a C₆₋₁₂aryloxyC₁₋₈alkyl group such as an optionally substituted phenyloxymethyl, phenyloxyethyl, 1-naphthyloxymethyl, or 2-naphthyloxymethyl group; an optionally substituted C₁₋₈alkanoyloxyC₁₋₈alkyl group, such as a pivaloyloxymethyl, propionyloxyethyl or propionyloxypropyl group; or a C₆₋₁₂aroyloxyC₁₋₈alkyl group such as an optionally substituted benzoyloxyethyl or benzoyloxypropyl group. Optional substituents present on the Alk⁴ group include R^{10a} atoms and groups as described above.

When Alk³ is present in or as a substituent it may be for example a methylene, ethylene, n-propylene, i-propylene, n-butylene, i-butylene, s-butylene, t-butylene, ethenylene, 1-propenylene, 2-propenylene, 1-butenylene, 2-butenylene, 3-butenylene, ethynylene, 1-propynylene, 2-propynylene, 1-butynylene, 2-butynylene or 3-butynylene chain, optionally interrupted by one, two, or three —O— or —S— atoms or —S(O)—, —S(O)₂— or —N(R¹²)—, e.g. —N(CH₃)— groups.

Aryl or heteroaryl groups represented by the groups R^{10a} or R¹¹ include mono- or bicyclic optionally substituted C₆₋₁₂ aromatic or C₁₋₉ heteroaromatic groups as described above for the group G. The aromatic and heteroaromatic groups may be attached to the group G in compounds of formula (1) by any carbon or hetero e.g. nitrogen atom as appropriate.

When —NHet¹ forms part of a substituent R¹⁰ each may be for example an optionally substituted pyrrolidinyl, pyrazolidinyl, piperazinyl, morpholinyl, thiomorpholinyl, piperidinyl or thiazolidinyl group. Optional substituents which may be present on —NHet¹ include those substituents described above when G is a heterocycloaliphatic group.

Particularly useful atoms or groups represented by R¹⁰ include fluorine, chlorine, bromine or iodine atoms, or C₁₋₆alkyl, e.g. methyl, ethyl, n-propyl, i-propyl, n-butyl or t-butyl, optionally substituted phenyl, pyridyl, pyrimidinyl, pyrrolyl, furyl, thiazolyl, or thienyl, C₁₋₆hydroxyalkyl, e.g. hydroxymethyl or hydroxyethyl, carboxyC₁₋₆alkyl, e.g. carboxyethyl, C₁₋₆alkylthio e.g. methylthio or ethylthio, carboxyC₁₋₆alkylthio, e.g. carboxymethylthio, 2-carboxyethylthio or 3-carboxypropylthio, C₁₋₆alkoxy, e.g. methoxy or ethoxy, hydroxyC₁₋₆alkoxy, e.g. 2-hydroxyethoxy, optionally substituted phenoxy, pyridyloxy, thiazolyoxy, phenylthio or pyridylthio, C₃₋₇cycloalkyl, e.g. cyclobutyl, cyclopentyl, C₅₋₇cycloalkoxy, e.g. cyclopentyloxy, haloC₁₋₆alkyl, e.g. trifluoromethyl, haloC₁₋₆alkoxy, e.g. trifluoromethoxy, C₁₋₆alkylamino, e.g. methylamino or ethylamino, amino (—NH₂), aminoC₁₋₆alkyl, e.g. aminomethyl or aminoethyl, C₁₋₆dialkylamino, e.g. dimethylamino or diethylamino, C₁₋₆alkylaminoC₁₋₆alkyl, e.g. ethylaminoethyl, C₁₋₆dialkylaminoC₁₋₆alkyl, e.g. diethylaminoethyl, aminoC₁₋₆alkoxy, e.g. aminoethoxy, C₁₋₆alkylaminoC₁₋₆alkoxy, e.g. methylaminoethoxy, C₁₋₆dialkylaminoC₁₋₆alkoxy, e.g. dimethylaminoethoxy, diethylaminoethoxy, diisopropylaminoethoxy, or dimethylaminopropoxy, imido, such as phthalimido or naphthalimido, e.g. 1,8-naphthalimido, nitro, cyano, amidino, hydroxyl (—OH), formyl [HC(O)—], carboxyl (—CO₂H), —CO₂Alk⁴ [where Alk⁴is as defined above], C₁₋₆ alkanoyl e.g. acetyl, optionally substituted benzoyl, thiol (—SH), thioC₁₋₆alkyl, e.g. thiomethyl or thioethyl, —SC(═NH)NH₂, sulphonyl (—SO₃H), C₁₋₆alkylsulphonyl, e.g. methylsulphonyl, aminosulphonyl (—SO₂NH₂), C₁₋₆alkylaminosulphonyl, e.g. methylaminosulphonyl or ethylaminosulphonyl, C₁₋₆dialkylaminosulphonyl, e.g. dimethylaminosulphonyl or diethylaminosulphonyl, phenylaminosulphonyl, carboxamido (—CONH₂), C₁₋₆alkylaminocarbonyl, e.g. methylaminocarbonyl or ethylaminocarbonyl, C₁₋₆dialkylaminocarbonyl, e.g. dimethylaminocarbonyl or diethylaminocarbonyl, aminoC₁₋₆alkylaminocarbonyl, e.g. aminoethylaminocarbonyl, C₁₋₆dialkylaminoC₁₋₆alkylaminocarbonyl, e.g. diethylaminoethylaminocarbonyl, aminocarbonylamino, C₁₋₆alkylaminocarbonylamino, e.g. methylaminocarbonylamino or ethylaminocarbonylamino, C₁₋₆dialkylaminocarbonylamino, e.g. dimethylaminocarbonylamino or diethylaminocarbonylamino, C₁₋₆alkylaminocabonylC₁₋₆alkylamino, e.g. methylaminocarbonylmethylamino, aminothiocarbonylamino, C₁₋₆alkylaminothiocarbonylamino, e.g. methylaminothiocarbonylamino or ethylaminothiocarbonylamino, C₁₋₆dialkylaminothiocarbonylamino, e.g. dimethylaminothiocarbonylamino or diethylaminothiocarbonylamino, C₁₋₆alkylaminothiocarbonylC₁₋₆alkylamino, e.g. ethylaminothiocarbonylmethylamino, —CONHC(═NH)NH₂, C₁₋₆alkylsulphonylamino, e.g. methylsulphonylamino or ethylsulphonylamino, C₁₋₆dialkylsulphonylamino, e.g. dimethylsulphonylamino or diethylsulphonylamino, optionally substituted phenylsulphonylamino, aminosulphonylamino (—NHSO₂NH₂), C₁₋₆alkylaminosulphonylamino, e.g. methylaminosulphonylamino or ethylaminosulphonylamino, C₁₋₆dialkylaminosulphonylamino, e.g. dimethylaminosulphonylamino or diethylaminosulphonylamino, optionally substituted morpholinesulphonylamino or morpholinesulphonylC₁₋₆alkylamino, optionally substituted phenylaminosulphonylamino, C₁₋₆alkanoylamino, e.g. acetylamino, aminoC₁₋₆alkanoylamino e.g. aminoacetylamino, C₁₋₆dialkylaminoC₁₋₆alkanoylamino, e.g. dimethylaminoacetylamino, C₁₋₆alkanoylaminoC₁₋₆alkyl, e.g. acetylaminomethyl, C₁₋₆alkanoylaminoC₁₋₆alkylamino, e.g. acetamidoethylamino, C₁₋₆alkoxycarbonylamino, e.g. methoxycarbonylamino, ethoxycarbonylamino or t-butoxycarbonylamino or optionally substituted benzyloxy, pyridylmethoxy, thiazolylmethoxy, benzyloxycarbonylamino, benzyloxycarbonylaminoC₁₋₆alkyl e.g. benzyloxycarbonylaminoethyl, benzothio, pyridylmethylthio or thiazolylmethylthio groups.

Where desired, two R¹⁰ substituents may be linked together to form a cyclic group such as a cyclic ether, e.g. a C₁₋₆alkylenedioxy group such as methylenedioxy or ethylenedioxy.

It will be appreciated that where two or more R¹⁰ substituents are present, these need not necessarily be the same atoms and/or groups. In general, the substituent(s) may be present at any available ring position in the aromatic or heteroaromatic group represented by the group G.

Optionally substituted carbon-linked bicyclic heteroaromatic groups represented by B and/or B′ include for example eight to thirteen-membered fused ring heteroaromatic groups containing one, two, three or four heteroatoms selected from oxygen sulphur or nitrogen atoms where in each case the heteroaromatic group is linked to the remainder of the compound of the invention via a carbon atom.

Particular examples of carbon-linked bicyclic heteroaromatic groups include benzofuryl, [2,3-dihydro]benzofuryl, benzothienyl, [2,3-dihydro]benzothienyl, benzotriazolyl, indolyl, indolinyl, indazolinyl, benzimidazolyl, imidazo[1,2-a]pyridyl, benzothiazolyl, benzoxazolyl, benzisoxazolyl, benzopyranyl, [3,4-dihydro]benzopyranyl, quinazolinyl, quinoxalinyl, naphthyridinyl, pyrido[3,4-b]pyridyl, pyrido[3,2-b]pyridyl, pyrido[4,3-b]pyridyl, quinolinyl, isoquinolinyl, cinnolinyl, phthalazinyl, 5,6,7,8-tetrahydroquinolinyl, 5,6,7,8-tetrahydroisoquinolinyl, phthalimidyl, naphthalimidyl such as 1,8-naphthalimidyl, pyrazolo[4,3-d]pyrimidinyl, furo[3,2-d]pyrimidinyl, thieno[3,2-d]pyrimidinyl, pyrrolo[3,2-d]pyrimidinyl, pyrazolo[3,2-b]pyridinyl, furo[3,2-b]pyridinyl, thieno[3,2-b]pyridinyl, pyrrolo[3,2-b]pyridinyl, thiazolo[3,2-a]pyyridinyl, pyrido[1,2-a]pyrimidinyl, tetrahydroimidazo[1,2-a]pyrimidinyl and dihydroimidazo[1,2-a]pyrimidinyl groups.

The carbon-linked bicyclic heterocyclic group represented by B and/or B′ may be optionally substituted on any available carbon or nitrogen atom. One, two, three or more of the same or different substituents (R¹³) may be present and each substituent may be selected from an atom or group -L⁴(Alk⁵)ₜL⁵(R¹⁴)ᵤ in which L⁴ and L⁵ which may be the same or different is each a covalent bond or a linker atom or group, t is zero or the integer 1, u is an integer 1, 2 or 3, Alk⁵ is a straight or branched C₁₋₆alkylene, C₂₋₆alkenylene or C₂₋₆alkynylene chain, optionally interrupted by one, two or three —O— or —S— atoms or —S(O)ₙ— [where n is an integer 1 or 2] or —N(R¹²)— groups and R¹⁴ is a hydrogen or halogen atom or a group selected from alkyl, —OR¹⁵ [where R¹⁵ is a hydrogen atom or an optionally substituted alkyl group], —SR¹⁵, —NR¹⁵R¹⁶ [ where R¹⁶ is as just defined for R¹⁵ and may be the same or different], —NO₂, —N₃, —CN, —CO₂R¹⁵, —SO₃H, —SOR¹⁵, —SO₂R¹⁵, —SO₃R¹⁵, —OCO₂R¹⁵, —CONR¹⁵R¹⁶, —OCONR¹⁵R¹⁶, —CSNR¹⁵R¹⁶, —COR¹⁵, —OCOR¹⁵, —N(R¹⁵)COR¹⁶, —N(R¹⁵)CSR¹⁶, —SO₂N(R¹⁵)(R¹⁶), —N(R¹⁵)SO₂R¹⁶, —N(R¹⁵)CON(R¹⁶)(R¹⁷) [where R¹⁷ is as just defined for R¹⁵], —N(R¹⁵)CSN(R¹⁶)(R¹⁷), —N(R¹⁵)SO₂N(R¹⁶)(R¹⁷), aryl or a heteroaryl group.

When L⁴ and/or L⁵ is present in these substituents as a linker atom or group it may be any divalent linking atom or group as previously defined for L¹.

When R¹⁴, R¹⁵, R¹⁶ and/or R¹⁷ is present as an optionally substituted alkyl group it may be an optionally substituted straight or branched C₁₋₆alkyl group as previously generally and particularly defined or an optionally substituted C₃₋₈cycloalkyl group as previously defined for R⁵. Optional substituents which may be present on such groups include those optional substituents as previously described.

Additionally when the groups R¹⁵ and R¹⁶ or R¹⁶ and R¹⁷ are both alkyl groups these groups may be joined, together with the N atom to which they are attached, to form a heterocyclic ring. Such heterocyclic rings may be optionally interrupted by a further heteroatom or heteroatom containing group selected from —O—, —S—, —N(R¹⁵)—, —C(O)— or —C(S)— groups. Particular examples of such heterocyclic rings include piperidinyl, pyrazolidinyl, morpholinyl, thiomorpholinyl, pyrrolidinyl, imidazolidinyl and piperazinyl rings.

When Alk⁵ is present in or as a substituent it may be a chain as defined hereinbefore for the chain Alk³.

Aryl and heteroaryl groups represented by R¹⁴ include those aromatic and heteroaromatic groups as previously described in relation to the group G. Optional substituents which may be present on these groups include those optional substituents described hereinbefore when G is an aromatic or heteroaromatic group.

Examples of the substituents represented by -L⁴(Alk⁵)ₜL⁵(R¹⁴)ᵤ when present as R¹³ substituents on heterocycles represented by the group B in compounds of the invention include atoms or groups -L⁴Alk⁵R¹⁴, -L⁴Alk⁵R¹⁴, -Alk⁵L⁵R¹⁴, -L⁴R¹⁴ and -Alk⁵R¹⁴ wherein L⁴, Alk⁵, L⁵ and R¹⁴ are as defined above. Particular examples of such substituents include -L⁴CH₂L⁵R¹⁴, -L⁴CH(CH₃)L⁵R¹⁴, -L⁴CH(CH₂)₂L⁵R¹⁴, -L⁴CH₂R¹⁴, -L⁴CH(CH₃)R¹⁴, -L⁴(CH₂)₂R¹⁴, —CH₂R¹⁴, —CH(CH₃)R¹⁴, —(CH₂)₂R¹⁴, —CHCHR¹⁴, —CH₂CHCHR¹⁴, —CCR¹⁴, —CH₂CCR¹⁴ and —R¹⁴ groups.

Thus R¹³ substituents which may be present on carbon-linked bicyclic heterocyclic group represented by B and/or B′ in compounds of the invention may include for example one, two, three or more halogen atoms, e.g. fluorine, chlorine, bromine or iodine atoms, and/or C₁₋₆alkyl, e.g. methyl, ethyl, n-propyl, i-propyl, n-butyl or t-butyl, C₂₋₆alkenyl, e.g. —CHCH₂, —CHCHCH₃, —CH₂CHCH₂ or —CH₂CHCHCH₃, C₂₋₆alkynyl, e.g. —CCH, —CCCH₃, —CH₂CCH or —CH₂CCCH₃, C₃₋₇cycloalkyl, e.g. cyclobutyl, cyclopentyl, optionally substituted aryl, e.g. optionally substituted phenyl, optionally substituted heteroaryl, e.g. optionally substituted pyridyl, pyrimidinyl, pyrrolyl, furyl, thiazolyl, or thienyl, optionally substituted arylC₁₋₆alkyl or heteroarylC₁₋₆alkyl, e.g. optionally substituted benzyl, pyridylmethylenyl, thiazolmethylenyl, optionally substituted aryloxy, e.g. phenoxy, optionally substituted heteroaryloxy, e.g. optionally substituted pyridyloxy, thiazolyoxy, optionally substituted arylthio or heteroarylthio, e.g. optionally substituted phenylthio or pyridylthio, optionally substituted arylC₁₋₆alkyloxy or heteroarylC₁₋₆alkyloxy, e.g. optionally substituted benzyloxy, pyridylmethoxy, thiazolylmethoxy, optionally substituted arylC₁₋₆alkylamino or heteroarylC₁₋₆alkylamino, e.g. optionally substituted benzylamino, pyridylmethylamino, thiazolylmethylamino, optionally substituted arylC₁₋₆alkylthio or heteroarylC₁₋₆alkylthio, e.g. benzothio, pyridylmethylthio or thiazolylmethylthio C₁₋₆hydroxyalkyl, e.g. hydroxymethyl, hydroxyethyl or —C(OH)(CF₃)₂, carboxyC₁₋₆alkyl, e.g. carboxyethyl, C₁₋₆alkylthio e.g. methylthio or ethylthio, carboxyC₁₋₆alkylthio, e.g. carboxymethylthio, 2-carboxyethylthio or 3-carboxypropylthio, C₁₋₆alkoxy, e.g. methoxy or ethoxy, hydroxyC₁₋₆alkoxy, e.g. 2-hydroxyethoxy, haloC₁₋₆alkyl, e.g. —CF₃, —CHF₂, CH₂F, haloC₁₋₆alkoxy, e.g. —OCF₃, —OCHF₂, —OCH₂F, C₁₋₆alkylamino, e.g. methylamino or ethylamino, amino (—NH₂), aminoC₁₋₆alkyl, e.g. aminomethyl or aminoethyl, C₁₋₆dialkylamino, e.g. dimethylamino or diethylamino, C₁₋₆alkylaminoC₁₋₆alkyl, e.g. ethylaminoethyl, C₁₋₆ dialkylaminoC₁₋₆alkyl, e.g. diethylaminoethyl, aminoC₁₋₆alkoxy, e.g. aminoethoxy, C₁₋₆alkylaminoC₁₋₆alkoxy, e.g. methylaminoethoxy, C₁₋₆dialkylaminoC₁₋₆alkoxy, e.g. dimethylaminoethoxy, diethylaminoethoxy, diisopropylaminoethoxy, or dimethylaminopropoxy, nitro, azido, cyano, amidino, hydroxyl (—OH), formyl [HC(O)—], carboxyl (—CO₂H), —CO₂Alk⁶ [where Alk⁶ is as defined above for Alk⁴], C₁₋₆ alkanoyl e.g. acetyl, thiol (—SH), thioC₁₋₆alkyl, e.g. thiomethyl or thioethyl, sulphonyl (—SO₃H), C₁₋₆alkylsulphonyl, e.g. methylsulphonyl, aminosulphonyl (—SO₂NH₂), C₁₋₆ alkylaminosulphonyl, e.g. methylaminosulphonyl or ethylaminosulphonyl, C₁₋₆dialkylaminosulphonyl, e.g. dimethylaminosulphonyl or diethylaminosulphonyl, phenylaminosulphonyl, carboxamido (—CONH₂), C₁₋₆alkylaminocarbonyl, e.g. methylaminocarbonyl or ethylaminocarbonyl, C₁₋₆dialkylaminocarbonyl, e.g. dimethylaminocarbonyl or diethylaminocarbonyl, aminoC₁₋₆alkylaminocarbonyl, e.g. aminoethylaminocarbonyl, C₁₋₆dialkylaminoC₁₋₆alkylaminocarbonyl, e.g. diethylaminoethylaminocarbonyl, aminocarbonylamino, C₁₋₆alkylaminocarbonylamino, e.g. methylaminocarbonylamino or ethylaminocarbonylamino, C₁₋₆dialkylaminocarbonylamino, e.g. dimethylaminocarbonylamino or diethylaminocarbonylamino, C₁₋₆alkylaminocabonylC₁₋₆alkylamino, e.g. methylaminocarbonylmethylamino, aminothiocarbonylamino, C₁₋₆alkylaminothiocarbonylamino, e.g. methylaminothiocarbonylamino or ethylaminothiocarbonylamino, C₁₋₆dialkylaminothiocarbonylamino, e.g. dimethylaminothiocarbonylamino or diethylaminothiocarbonylamino, C₁₋₆alkylaminothiocarbonylC₁₋₆alkylamino, e.g. ethylaminothiocarbonylmethylamino, C₁₋₆alkylsulphonylamino, e.g. methylsulphonylamino or ethylsulphonylamino, C₁₋₆dialkylsulphonylamino, e.g. dimethylsulphonylamino or diethylsulphonylamino, aminosulphonylamino (—NHSO₂NH₂), C₁₋₆alkylaminosulphonylamino, e.g. methylaminosulphonylamino or ethylaminosulphonylamino, C₁₋₆dialkylaminosulphonylamino, e.g. dimethylaminosulphonylamino or diethylaminosulphonylamino, C₁₋₆alkanoylamino, e.g. acetylamino, aminoC₁₋₆alkanoylamino e.g. aminoacetylamino, C₁₋₆dialkylamino-C₁₋₆alkanoylamino, e.g. dimethylaminoacetylamino, C₁₋₆alkanoylaminoC₁₋₆alkyl, e.g. acetylaminomethyl, C₁₋₆alkanoylaminoC₁₋₆alkylamino, e.g. acetamidoethylamino, C₁₋₆alkoxycarbonylamino, e.g. methoxycarbonylamino, ethoxycarbonylamino or t-butoxycarbonylamino groups.

Where desired, two R¹³ substituents may be linked together to form a cyclic group such as a cyclic ether, e.g. a C₁₋₆alkylenedioxy group such as methylenedioxy or ethylenedioxy.

The presence of certain substituents in the compounds of formula (1) may enable salts of the compounds to be formed. Suitable salts include pharmaceutically acceptable salts, for example acid addition salts derived from inorganic or organic acids, and salts derived from inorganic and organic bases.

Acid addition salts include hydrochlorides, hydrobromides, hydrolodides, alkylsulphonates, e.g. methanesulphonates, ethanesulphonates, or isothionates, arylsulphonates, e.g. p-toluenesulphonates, besylates or napsylates, phosphates, sulphates, hydrogen sulphates, acetates, trifluoroacetates, propionates, citrates, maleates, fumarates, malonates, succinates, lactates, oxalates, tartrates and benzoates.

Salts derived from inorganic or organic bases include alkali metal salts such as sodium, lithium or potassium salts, alkaline earth metal salts such as magnesium or calcium salts, and organic amine salts such as ammonia, morpholine, piperidine, dimethylamine, trimethylamine, diethylamine, triethylamine, cyclohexylamine or tris(hydroxymethyl)aminomethane salts.

Particularly useful salts of compounds according to the invention include pharmaceutically acceptable salts, especially base addition pharmaceutically acceptable salts.

One particular class of compounds of formulae (1) and (1c) is that wherein the group G′ has the formula (1b) in which the furanose sugar is preferably in the β-configuration, preferably the β-D-configuration, most preferably the β-D-ribofuranose configuration. Particularly useful compounds of the invention include those where Y′ is a hydroxyl (—OH) group.

A particularly useful group of compounds according to the invention has the formula (2a): 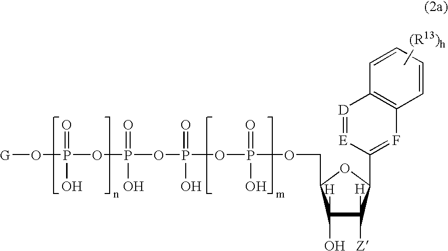
wherein:
D, E and F is each a carbon or nitrogen atom provided that no more than two of D, E and F is a nitrogen atom;
Z′ is a hydroxyl (—OH), amino (—NH₂) or azido (—N₃) group;
G, is as previously defined for compounds of formula (1);
n and m is each as previously defined for compounds of formula (1);
the ribose sugar is of natural β-D configuration as shown;
h is zero or the integer 1, 2, 3 or 4;
R¹³ is an optional substituent as previously defined which may be on any available carbon atom of the heterocyclic ring B′;
   and the salts, solvates, hydrates and N-oxides thereof.

A further particularly useful group of compounds according to the invention has the formula (2b): 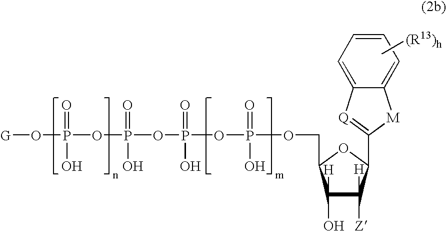
wherein:
Q is a N atom or a CH or C(R¹³) group;
M is an oxygen or sulphur atom or an NH or N(R¹³) group;
Z′ is a hydroxyl (—OH), amino (—NH₂) or azido (—N₃) group;
G is as previously defined for compounds of formula (1);
n and m is each as previously defined for compounds of formula (1);
the ribose sugar is of natural β-D configuration as shown;
h is zero or the integer 1, 2, 3 or 4;
R¹³ is an optional substituent as previously defined which may be on any available carbon or nitrogen atom of the heterocyclic ring B′;
   and the salts, solvates, hydrates and N-oxides thereof.

In compounds of formula (1), (2a) and (2b) Z′ is preferably a hydrogen atom or most preferably a hydroxyl (—OH) group.

In one particularly useful class of compounds of formula (1), (1c), (2a) and (2b) G is preferably a hydrogen atom or an optionally substituted aliphatic, heteroaliphatic, cycloaliphatic, polycycloaliphatic, aromatic or heteroaromatic group or a group of formula (1a): 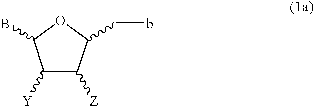
as hereinbefore generally and particularly defined.

When G is a group of formula (1a) Y and Z is each preferably a hydrogen atom or a hydroxyl group. Most preferably Y and Z is each a hydroxyl (—OH) group.

When G is an optionally substituted aliphatic group it may in particular be an optionally substituted C₁₋₆alkylene, C₂₋₆alkenylene or C₂₋₆alkynylene group as hereinbefore defined.

When G is an optionally substituted heteroaliphatic group it may in particular be an optionally substituted aliphatic group as just defined but additionally containing one, two, three or four L¹ atoms or groups where L¹ is preferably an —O— or —S— atom or an —N(R¹)—, especially —N(CH₃)— group.

Particularly preferred optional substituents which may be present on aliphatic and heteroaliphatic groups represented by G include one, two or three substituents where each substituent may be the same or different and is selected from halogen atoms, e.g. fluorine, chlorine, bromine or iodine atoms, or hydroxyl (—OH), C₁₋₆alkoxy, e.g. methoxy or ethoxy, C₁₋₆haloalkoxy, e.g. trifluoromethoxy or difluoromethoxy, thiol (—SH), C₁₋₆alkylthio e.g. methylthio or ethylthio or optionally substituted cycloaliphatic, especially cyclopropyl, cyclobutyl, cyclopentyl or cyclohexyl, heterocycloaliphatic, especially tetrahydrofuranyl, dihydrofuranyl or tetrahydropyranyl, aromatic, especially phenyl or heteroaromatic, especially thienyl, furanyl, pyridyl or pyrimidinyl groups.

When G is an optionally substituted cycloaliphatic group it may in particular be an optionally substituted C₃₋₇cycloalkyl or C₃₋₇cycoalkenyl group as hereinbefore defined.

When G is an optionally substituted polycycloaliphatic group it may in particular be an optionally substituted C₇₋₁₀bi- or tricycloalkyl group.

Particular examples of cycloaliphatic and polycycloaliphatic groups represented by the group G include optionally substituted cyclopropyl, cyclobutyl, cyclopentyl, cyclohexyl, cycloheptyl, 2-cyclobuten-1-yl, 2-cyclopenten-1-yl, 3-cyclopenten-1-yl, adamantyl, norbornyl, and norbornenyl, groups.

Particularly preferred optional substituents which may be present on cycloaliphatic and polycycloaliphatic groups represented by the group G include one, two, three or more substituents selected from halogen atoms, or C₁₋₆alkyl, e.g. methyl or ethyl, haloC₁₋₆alkyl, e.g. halomethyl or haloethyl such as difluoromethyl or trifluoromethyl, optionally substituted by hydroxyl, e.g. —C(OH)(CF₃)₂, hydroxyl (—OH), C₁₋₆alkoxy, e.g. methoxy or ethoxy, haloC₁₋₆alkoxy, e.g. halomethoxy or haloethoxy such as difluoromethoxy or trifluoromethoxy, thiol (—SH), C₁₋₆alkylthiol, e.g. methylthiol or ethylthiol or optionally substituted cycloalphatic, heterocycloaliphatic, aromatic or heteroaromatic groups.

Optionally substituted cycloaliphatic, heterocycloaliphatic, aromatic or heteroaromatic groups when present as optional substituents on the cycloaliphatic and polycycloaliphatic groups represented by the group G include those optionally substituted cycloaliphatic, heterocycloaliphatic, aromatic or heteroaromatic groups as described hereinbefore in relation to optional substituents on preferred aliphatic or heteroaliphatic groups represented by G.

Particularly useful R¹³ substituents when present in compounds of formulae (1), (1c), (2a) or (2b) include halogen atoms, especially fluorine or chlorine atoms, or straight or branched C₁₋₆alkyl groups, especially methyl, ethyl, propyl or isopropyl groups, C₂₋₆alkenyl, especially —CHCH₂ and —CHCHCH₃, C₁₋₆alkynyl, especially —CCH and —CCCH₃, C₃₋₈cycloalkyl groups, especially cyclopropyl, cyclobutyl, cyclopentyl or cyclohexyl groups, haloC₁₋₆alkyl groups, especially halomethyl groups, especially —CF₃ and —CHF₂ groups, C₁₋₆alkoxy groups, especially methoxy or ethoxy groups, haloC₁₋₆alkoxy groups, especially halomethoxy groups, most especially —OCF₃ or —OCHF₂ groups, C₁₋₆alkylthiol groups, especially methylthiol or ethylthiol groups, —CN, —CO₂Alk⁶, especially —CO₂CH₃ and —CO₂C(CH₃)₃, —NO₂, amino (—NH₂), substituted amino (—NR¹⁵R¹⁶), especially —NHCH₃ and —N(CH₃)₂, —N(R¹⁵)COR¹⁶, especially —NHCOCH₃ and —COR¹⁵, especially —COCH₃ groups. In one particular group of compounds of this type R¹³ is selected from fluorine, chlorine, methyl, methoxy or —CF₃.

The particularly useful R¹³ substituents as just described also represent particularly useful optional substituents in general on aromatic and heteroaromatic groups when present in compounds of the invention.

In one preferred group of compounds of formulae (1), (1c), (2a) or (2b) h is zero or the integer 1 or 2.

In one preferred class of compounds of formulae (2a) D and E is each a carbon atom and F is a nitrogen atom.

In another preferred class of compounds of formulae (2a) D and F is each a carbon atom and E is a nitrogen atom.

In another preferred class of compounds of formulae (2a) E and F is each a carbon atom and D is a nitrogen atom.

In another particularly preferred class of compounds of formulae (2a) E and F is each a nitrogen atom and D is a carbon atom.

In one preferred class of compounds of formulae (2b) M is an oxygen atom and Q is a CH or C(R¹³) group, where R¹³ is preferably a —CH₃ group.

In another preferred class of compounds of formulae (2b) M is an sulphur atom and Q is a CH or C(R¹³) group, where R¹³ is preferably a —CH₃ group.

In another preferred class of compounds of formulae (2b) M is an NH or N(R¹³), especially N(CH₃) group and Q is a CH or C(R¹³) group, where R¹³ is preferably a —CH₃ group.

In a particularly preferred class of compounds of formulae (2b) Q is a N atom and M is an oxygen or sulphur atom or NH or N(R¹³a), especially N(CH₃) group.

In another particularly preferred class of compounds of formulae (1), (1c), (2a) and (2b) G is a hydrogen atom. In this class 6of compounds m is preferably the integer 1 and n is preferably zero.

In another preferred class of compounds of formulae (1), (1c), (2a) and (2b) G is a nucleoside of formula (1a) in which the furanose sugar is preferably in the β-configuration, preferably the β-D-configuration, most preferably the β-D-ribofuranose configuration as shown in formula (2c): 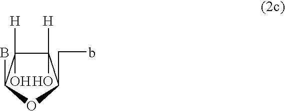
where the letter b indicates the point of attachment to the remainder of the compound of formula (1), (1c), (2a) or (2b). In this class of compounds m and n is each preferably the integer 1. In one preferred group of compounds of this class the heterocycle B is a group of formula (2d): 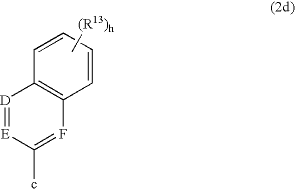
wherein h is as previously defined, c represents the point of attachment to the remainder of the molecule of formula (2c) and D, E, F and R¹³ are as previously generally defined and particularly defined in relation to compounds of formula (2a). In another preferred group of compounds of this class the heterocycle B is a group of formula (2e): 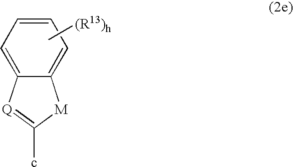
wherein h and c are as previously defined and Q, M and R¹³ are as previously generally defined and particularly defined in relation to compounds of formula (2b).

A most preferred group of compounds of the invention is that where G is a group of formula (1a) and G′ is a group of formula (1b) in which the optionally substituted heterocycles B and B′ is each a group of formula (2d) or is each a group of formula (2e). Especially preferred is a compound of formula (2a) in which G is a group of formula (2c) in which B is a group of formula (2d), most preferably where B and B′ are identical or a compound of formula (2b) in which G is a group of formula (2c) in which B is a group of formula (2e), most preferably where B and B′ are identical.

A further most preferred group of compounds of the invention is that where G is a group of formula (1a), most preferably the β-D-ribofuranose configuration and G′ is a group of formula (1b), most preferably the β-D-ribofuranose configuration in which B is an optionally substituted heterocycle of formula (2d) and B′ is an optionally substituted heterocycle of formula (2e).

In another preferred group of compounds of formulae (1), (2a) and (2b) G is a C₁₋₆alkyl group, especially a methyl, ethyl, propyl, isopropyl or t-butyl group, a haloC₁₋₆alkyl group, especially a trifluoromethyl or difluoromethyl group, a C₁₋₆heteroalkyl group, especially ethyloxymethyl, propyloxymethyl or butyloxymethyl group, an optionally substituted C₃₋₇heterocycloalkylC₁₋₆alkyl group, especially an optionally substituted tetrahydrofuranylmethyl or dihydrofuranylmethyl group, an optionally substituted C₆₋₁₀arylC₁₋₆alkyl group, especially an optionally substituted benzyl or phenylethyl group, an optionally substituted C₁₋₉heteroarylC₁₋₆alkyl group, especially an optionally substituted pyridylmethyl, thienylmethyl, furanylmethyl or pyrimidinylmethyl group, an optionally substituted C₃₋₇cycloalkylC₁₋₆alkyl group, especially an optionally substituted cyclopropylmethyl, cyclopentylmethyl or cyclohexylmethyl group, an optionally substituted C₃₋₇heterocycloalkyl group, especially an optionally substituted morpholinyl, thiomorpholinyl, piperidinyl, piperazinyl or pyrrolidinyl group, an optionally substituted C₆₋₁₀aromatic group, especially an optionally substituted phenyl group or an optionally substituted C₁₋₁₉heteroaromatic group, especially an optionally substituted pyridyl, pyrimidinyl, thienyl or furanyl group.

In one most preferred group of compounds of formulae (1), (2a) and (2b) G is a C₁₋₆alkyl group, especially a methyl, ethyl, propyl, isopropyl or t-butyl group, a haloC₁₋₆alkyl group, especially a trifluoromethyl or difluoromethyl group, a C₁₋₆heteroalkyl group, especially ethyloxymethyl, propyloxymethyl or butyloxymethyl group, an optionally substituted C₃₋₇heterocycloalkylC₁₋₆alkyl group, especially an optionally substituted tetrahydrofuranylmethyl or dihydrofuranylmethyl group, an optionally substituted C₆₋₁₀arylC₁₋₆alkyl group, especially an optionally substituted benzyl or phenylethyl group, an optionally substituted C₁₋₆heteroarylC₁₋₆alkyl group, especially an optionally substituted pyridylmethyl, thienylmethyl, furanylmethyl or pyrimidinylmethyl group, an optionally substituted C₃₋₇cycloalkylC₁₋₆alkyl group, especially an optionally substituted cyclopropylmethyl, cyclopentylmethyl or cyclohexylmethyl group, an optionally substituted C₆₋₁₀aromatic group, especially an optionally substituted phenyl group or an optionally substituted C₁₋₉heteroaromatic group, especially an optionally substituted pyridyl, pyrimidinyl, thienyl or fturanyl group.

Particularly useful compounds according to the invention are:
(2R,3S,4R,5R)-3,4-Dihydroxy-5-benzothiazol-2-yl-tetrahydrofuran-2-ylmethyl triphosphate tris-ammonium salt;
(2R,3S,4R,5R)-3,4-Dihydroxy-5-(5-trifluoromethyl-benzothiazol-2-yl)-tetrahydrofuran-2-ylmethyl triphosphate tris-ammonium salt;
   and the free acid, other pharmaceutically acceptable salts, solvates, hydrates and N-oxides thereof.

Further particularly useful compounds according to the invention are:
(2R,3S,4R,5R)-3,4-Dihydroxy-5-(6-Chlorobenzothiazol-2-yl)-tetrahydrofuran-2-ylmethyl triphosphate tris-ammonium salt;
(2R,3S,4R,5R)-3,4-Dihydroxy-5-(5-fluorobenzothiazol-2-yl)-tetrahydrofuran-2-ylmethyl triphosphate tris-triethylammonium salt;
(2R,3S,4R,5R)-5-(6-Chloro-4-fluorobenzothiazol-2-yl)-3,4-dihydroxy-tetrahydrofuran-2-ylmethyl triphosphate tris-triethylammonium salt;
   and the free acid, other pharmaceutically acceptable salts, solvates, hydrates and N-oxides thereof.

The compounds of the invention may be prepared by a number of processes as generally described below and more specifically in the Examples hereinafter. In the following process description, the symbols G, G′, B, B′, m and n when used in the formulae depicted are to be understood to represent those groups described above in relation to formula (1) unless otherwise indicated. In the reactions described below, it may be necessary to protect reactive functional groups, for example hydroxy, amino, thio, phosphate or carboxy groups, where these are desired in the final product, to avoid their unwanted participation in the reactions. Conventional protecting groups may be used in accordance with standard practice [see, for example, Green, T. W. in “Protective Groups in Organic Synthesis”, John Wiley and Sons, 1999 and the Examples hereinafter]. In some instances, deprotection may be the final step in the synthesis of a compound of formula (1) and the processes according to the invention described hereinafter are to be understood to extend to such removal of protecting groups. For convenience the processes described below all refer to a preparation of a compound of formula (1) but clearly the description applies equally to the preparation of compounds of formulae (1c), (2a) or (2b).

Thus according to a further aspect of the invention a triphosphate of formula (1) [in which G is a hydrogen atom, n is zero and m is the integer 1] may be prepared by reaction of a compound of formula (3): 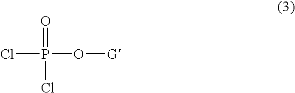
with a bis(tri-alkylammonium) pyrophosphate,—for example bis(tri-n-butylammonium) pyrophosphate in the presence of an organic base such as a trialkylamine, e.g. tributylamine in an anhydrous solvent e.g. an amide such as dimethylformamide or a sulfoxide such as dimethyl sulfoxide at a temperature from around 0 to 80° C., optionally followed by purification of the product (1) by for example ion exchange chromatography using a Luna C18 column eluting with an ammonium acetate buffer or aqueous formic acid or an anion exchange resin such as DEAE-A25 sephadex or Q-sepharose HP eluting with for example an ammonium salt buffer such as triethylammonium bicarbonate or ammonium bicarbonate. Such methods of preparing compounds of formula (1) are well known and may be found in for example Kovács, T., Tetrahedron Letters 1988, 29, 4525; Burgess, K. and Cook, D., Chem. Rev., 2000, 100, 2047.

Compounds of formula (1) in the ammonium ion salt form may be readily converted to other salt forms, for example the sodium salt form, by treatment with a cation exchange resin such as DOWEX®-50 in its Na⁺ form.

Intermediates of formula (3) may be obtained by the reaction of a compound of formula (1b) [where b now represents an —OH group] with a phosphorylating agent, for example phosphorous oxychloride in the presence of a weakly nucleophilic strong organic base such as Proton Sponge® in an anhydrous organic solvent, e.g. a phosphate such as trimethylphosphate or triethylphosphate at a low temperature, e.g. around 0° C. Such methods of preparing intermediates of formula (3) are well known and may be found in for example Yoshikawa, M. et al, Bull. Chem. Soc. Jpn. 1969, 42, 3505.

In a further procedure compounds of formula (1) in which G is other than a hydrogen atom and n and m is each the integer 1 may be prepared by reaction of the tri-n-butylammonium salt of a compound of formula (1) in which G is a hydrogen atom, n is zero and m is the integer 1 with an activating agent such as carbonyldiimidazole or a dialkyl carbodiimide, e.g. dicyclohexyl carbodiimide in the presence of an ammonium salt e.g. tri-n-butylammonium salt of a monophosphate of formula (4); 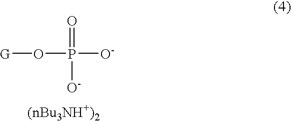
in a polar aprotic organic solvent such as a formamide e.g. dimethylformamide, a sulfoxide e.g. dimethylsulfoxide, a pyrrolidine e.g. N-methyl pyrrolidine, a phosphate e.g. triethylphosphate, a cyclic ether e.g. dioxane or an amine e.g. pyridine at a temperature from 0 to about 60° C. Purification of the resulting compounds of formula (1) may be achieved by any of the previously mentioned ion exchange procedures or by high performance liquid chromatography.

Methods of preparing monophosphates of formula (4) are well known in the art and may be found in for example Slotin, L. A., Synthesis, 1977, 737; Yoshikawa, M., et al, Bull. Chem. Soc. Jpn., 1969, 42, 3505.

In an alternative procedure for the preparation of compounds of formula (1) in which G is a group of formula (1a), G′ is a group of formula (1), n and m is each the integer 1 and the heterocyclic groups B and B′ are the same a diphosphate of formula (1d) [G is a hydrogen atom and n and m is each zero]; 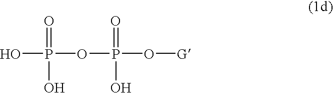
may be condensed with a further diphosphate of formula (1d) using any of the above mentioned activating agents.

Diphosphates of formula (1d) may be obtained from intermediate salts of formula (5): 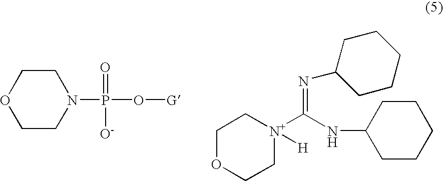
by reaction with a trialkylammonium phosphate salt e.g. bis(tri-n-butylammonium) orthophosphate in an anhydrous organic solvent such as anhydrous pyridine at around ambient temperature or in a phosphate solvent such as triethylphosphate at an elevated temperature e.g. around 50° C., as described by Moffatt, J. G. et al, J. Am. Chem. Soc. 1961, 83, 649-658 and Can. J. Chem. 1964, 42, 599-604.

Intermediates of formula (5) may be obtained by reaction of a salt of formula (6): 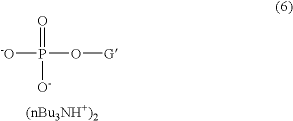
[or the free acid of a compound of formula (6)] with an activating agent such as a dialkyl carbodimide e.g. dicyclohexyl carbodimide or a carbonylimidazole, in the presence of an organic amine such as a cyclic amine e.g. morpholine [as depicted for formula (5)] in a solvent e.g. an alcohol such as t-butanol, i-propanol, ethanol or methanol in the presence of added water at a temperature from ambient to the reflux temperature.

In a further procedure for the preparation of compounds of formula (1) intermediates of formula (5) may be converted to triphosphates of formula (1) [in which G is a hydrogen atom, n is zero and m is the integer 1] by reaction with pyrophosphate (preferably as its tri-n-butylammonium salt) in an anhydrous polar aprotic solvent, for example dimethyl sulfoxide at for example ambient temperature.

Intermediate compounds of formula (1a) and (1b) [where b now represents an —OH group] may be prepared by the methods described hereinafter or by such well known methods as for example those of Bobek, M. et al, Collect. Czech Chem. Commun., 1968, 34, 247-252; El Khadem, H. S. et al, Carbohydrate Res., 1986, 153, 271-283; Szabó, I. F. et al, Acta Chimica Acad. Sci. Hung., 1982, 109, 229-236; Dudfield, P. J. et al, J. Chem. Soc., Perkin Trans. 1, 1999, 2937-42; He, W. et al, J. Chem. Soc., Perkin Trans. 1, 1998, 2425-34; Yokoyama, M. et al, J. Chem. Soc., Perkin Trans. 1, 1996, 2145-49, Gudmunsson, K. S. et al, Tetrahedron Lett., 1996, 37, 2365-8; Vismara, E. et al, Tetrahedron Lett., 1992, 33, 7575-8; Togo, H. et al, Tetrahedron Left., 1991, 32, 6559-62; Lopez Herrera, F. J. et al, J. Chem. Soc., Perkin Trans. 1, 1989, 2401-6; Frick, W. et al, Liebigs Ann. Chem., 1989, 565-70; Rao, S. P. et al, Tetrahedron Lett., 1988, 3537-40; Rosowsky, A. et al, Carbohydr. Res., 1988, 176, 47-58; Cupps, T. L. et at, J. Org. Chem., 1986, 51, 1058-64; Yokoyama, M. et al, Chem. Lett., 1994, 265-8; Yokoyama, M. et al, Synthesis, 1993, 517-20; Guianvarc'h, D. et al, Tetrahedron Lett., 2001, 647-50; Yokoyama, M. et al, J. Chem. Soc., Perkin Trans. 1, 1997, 29-33; Yokoyama, M. et al, Heteroat. Chem., 1995, 6, 189-93; Kim, G. et al, Tetrahedron Lett., 2000, 225-227; Maeba, I. et al, J. Org. Chem., 1988, 53, 1401-5 and Japanese patent application JP 07118268.

Where in the general processes described above intermediates such as those of formulae (1a), (1b), (1d), (3), (4), (5) and (6) are not commercially available or known in the literature they may be readily obtained from simpler known compounds by one or more standard synthetic methods employing substitution, oxidation, reduction or cleavage reactions. Particular substitution approaches include conventional alkylation, arylation, heteroarylation, acylation, thioacylation, halogenation, sulphonylation, nitration, formylation and coupling procedures. It will be appreciated that these methods may also be used to obtain or modify other compounds of formulae (1), (1c), (2a) and (2b) and also to further functionalize intermediates of formulae (1a), (1b), (1d), (3), (4), (5) and (6) where appropriate functional groups exist in these compounds.

Thus intermediates of formulae (1a), (1b), (1d), (3), (4), (5) and (6) and any other intermediates described herein required to obtain compounds of formula (1) may be prepared by methods known to those skilled in the art following procedures set forth in references such as Rodd's Chemistry of Carbon Compounds, Volumes 1-15 and Supplementals (Elsevier Science Publishers, 1989), Fieser and Fieser's Reagents for Organic Synthesis, Volumes 1-19 (John Wiley and Sons, 1999), Comprehensive Heterocyclic Chemistry, Ed. Katritzky et al, Volumes 1-8, 1984 and Volumes 1-11, 1994 (Pergamon), Comprehensive Organic Functional Group Transformations, Ed. Katritzky et al, Volumes 1-7, 1995 (Pergamon), Comprehensive Organic Synthesis, Ed. Trost and Flemming, Volumes 1-9, (Pergamon, 1991), Encyclopedia of Reagents for Organic Synthesis Ed. Paquette, Volumes 1-8 (John Wiley and Sons, 1995), Larock's Comprehensive Organic Transformations (VCH Publishers Inc., 1989) and March's Advanced Organic Chemistry (John Wiley and Sons, 5^{th} edition, 2001)

Compounds of the invention and intermediates thereto may be prepared by alkylation, arylation or heteroarylation. For example, compounds containing a -L³H or -L⁴H group (where L³ and L⁴ is each a linker atom or group) may be treated with an alkylating agent R⁹(Alk²)_{q}Z¹ or (R¹⁴)ᵤL⁵(Alk⁵)ₜZ¹ respectively in which Z¹ is a leaving atom or group such as a halogen atom, e.g. a fluorine, bromine, iodine or chlorine atom or a sulphonyloxy group such as an alkylsulphonyloxy, e.g. trifluoromethylsulphonyloxy or arylsulphonyloxy, e.g. p-toluenesulphonyloxy group.

The reaction may be carried out in the presence of a base such as a carbonate, e.g. caesium or potassium carbonate, an alkoxide, e.g. potassium t-butoxide, or a hydride, e.g. sodium hydride, in a dipolar aprotic solvent such as an amide, e.g. a substituted amide such as dimethylformamide or an ether, e.g. a cyclic ether such as tetrahydrofuran.

In another example, compounds containing a -L³H or -L⁴H or group as defined above may be functionalised by acylation or thioacylation, for example by reaction with one of the alkylating agents just described but in which Z¹ is replaced by a —C(O)Z², C(S)Z², —N(R²)COZ²or —N(R²)C(S)Z² group in which Z² is a leaving atom or group as described for Z¹. The reaction may be performed in the presence of a base, such as a hydride, e.g. sodium hydride or an amine, e.g. triethylamine or N-methylmorpholine, in a solvent such as a halogenated hydrocarbon, e.g. dichloromethane or carbon tetrachloride or an amide, e.g. dimethyl-formamide, at for example ambient temperature. Alternatively, the acylation may be carried out under the same conditions with an acid (for example one of the alkylating agents described above in which Z¹ is replaced by a —CO₂H group) in the presence of a condensing agent, for example a diimide such as 1-(3-dimethylaminopropyl)-3-ethylcarbodiimide or N,N′-dicyclohexylcarbodiimide, or a benzotriazole such as [O-(7-azabenzo-triazol-1-yl)-1,1,3,3-tetramethyluronium]hexafluorophosphate advantageously in the presence of a catalyst such as a N-hydroxy compound e.g. a N-hydroxytriazole such as 1-hydroxybenzotriazole. Alternatively the acid may be reacted with a chloroformate, for example ethylchloroformate, prior to the desired acylation reaction

In a further example compounds may be obtained by sulphonylation of a compound containing an —OH group by reaction with one of the above alkylating agents but in which Z¹ is replaced by a —S(O)Hal or —SO₂Hal group [in which Hal is a halogen atom such as chlorine atom] in the presence of a base, for example an inorganic base such as sodium hydride in a solvent such as an amide, e.g. a substituted amide such as dimethylformamide at for example ambient temperature.

In another example, compounds containing a -L³H or -L⁴H group as defined. above may be coupled with one of the alkylation agents just described but in which Z¹ is replaced by an —OH group in a solvent such as tetrahydrofuran in the presence of a phosphine, e.g. triphenylphosphine and an activator such as diethyl, diisopropyl- or dimethylazodicarboxylate.

In a further example, ester groups —CO₂R⁶, —CO₂Alk⁴, —CO₂Alk⁶ or —CO₂R¹⁵ in the compounds may be converted to the corresponding acid [—CO₂H] by acid- or base-catalysed hydrolysis depending on the nature of the groups R⁶, Alk⁴, Alk⁶ or R¹⁵. Acid- or base-catalysed hydrolysis may be achieved for example by treatment with an organic or inorganic acid, e.g. trifluoroacetic acid in an aqueous solvent or a mineral acid such as hydrochloric acid in a solvent such as dioxan or an alkali metal hydroxide, e.g. lithium hydroxide in an aqueous alcohol, e.g. aqueous methanol.

In a further example, —OR⁶ or —OR¹¹ groups [where R⁶ or R¹¹ each represents an alkyl group such as methyl group] in compounds of formula (1) may be cleaved to the corresponding alcohol —OH by reaction with boron tribromide in a solvent such as a halogenated hydrocarbon, e.g. dichloromethane at a low temperature, e.g. around −78° C.

Alcohol [—OH] groups may also be obtained by hydrogenation of a corresponding —OCH₂R³⁰ group (where R³⁰ is an aryl group) using a metal catalyst, for example palladium on a support such as carbon in a solvent such as ethanol in the presence of ammonium formate, cyclohexadiene or hydrogen, from around ambient to the reflux temperature. In another example, —OH groups may be generated from the corresponding ester [e.g. CO₂Alk² or CO₂R⁶] or aldehyde [—CHO] by reduction, using for example a complex metal hydride such as lithium aluminum hydride or sodium borohydride in a solvent such as methanol.

In another example, alcohol —OH groups in the compounds. may be converted to a corresponding —OR⁶ or —OR¹¹ group by coupling with a reagent R⁶OH or R¹¹OH in a solvent such as tetrahydrofuran in the presence of a phosphine, e.g. triphenylphosphine and an activator such as diethyl-, diisopropyl-, or dimethylazodicarboxylate.

Aminosulphonylamino [—NHSO₂NHR⁷] groups in the compounds may be obtained, in another example, by reaction of a corresponding amine [—NH₂] with a sulphamide R⁷NHSO₂NH₂ in the presence of an organic base such as pyridine at an elevated temperature, e.g. the reflux temperature.

In another example compounds containing a —NHCSR¹¹ or —CSNHR¹⁶, may be prepared by treating a corresponding compound containing a —NHCOR¹¹ or —CONHR¹⁶ group with a thiation reagent, such as Lawesson's Reagent or P₂S₅, in an anhydrous solvent, for example a cyclic ether such as tetrahydrofuran, at an elevated temperature such as the reflux temperature.

In a further example amine (—NH₂) groups may be alkylated using a reductive alkylation process employing an aldehyde and a borohydride, for example sodium triacetoxyborohyride or sodium cyanoborohydride, in a solvent such as halogenated hydrocarbon, e.g. dichloromethane, a ketone such as acetone, or an alcohol, e.g. ethanol, where necessary in the presence of an acid such as acetic acid at around ambient temperature.

In a further example, amine [—NH₂] groups in compounds of formula (1) may be obtained by hydrolysis from a corresponding imide by reaction with hydrazine in a solvent such as an alcohol, e.g. ethanol at ambient temperature.

In another example, a nitro [—NO₂] group may be reduced to an amine [—NH₂], for example by catalytic hydrogenation using for example hydrogen in the presence of a metal catalyst, for example palladium on a support such as carbon in a solvent such as an ether, e.g. tetrahydrofuran or an alcohol e.g. methanol, or by chemical reduction using for example a metal, e.g. tin or iron, in the presence of an acid such as hydrochloric acid.

In a further example amine (—CH₂NH₂) groups in compounds of formula (1) and intermediates thereto may be obtained by reduction of nitriles (—CN), for example by catalytic hydrogenation using for example hydrogen in the presence of a metal catalyst, for example palladium on a support such as carbon, or Raney® nickel, in a solvent such as an ether e.g. a cyclic ether such as tetrahydrofuran or an alcohol e.g. methanol or ethanol, optionally in the presence of ammonia solution at a temperature from ambient to the reflux temperature, or by chemical reduction using for example a metal hydride e.g. lithium aluminium hydride, in a solvent such as an ether e.g. a cyclic ether such as tetrahydrofuran, at a temperature from 0° C. to the reflux temperature.

Aromatic halogen substituents in the compounds may be subjected to halogen-metal exchange with a base, for example a lithium base such as n-butyl or t-butyl lithium, optionally at a low temperature, e.g. around −78° C., in a solvent such as tetrahydrofuran and then quenched with an electrophile to introduce a desired substituent. Thus, for example, a formyl group may be introduced by using dimethylformamide as the electrophile; a thiomethyl group may be introduced by using dimethyldisulphide as the electrophile. Aromatic acids may be generated by quenching aromatic Grignard reagents with carbon dioxide.

In another example, sulphur atoms in the compounds, for example when present in a linker group L³ or L⁴ may be oxidised to the corresponding sulphoxide or sulphone using an oxidising agent such as a peroxy acid, e.g. 3-chloroperoxybenzoic acid, in an inert solvent such as a halogenated hydrocarbon, e.g. dichloromethane, at around ambient temperature.

N-oxides of compounds of formula (1) may be prepared for example by oxidation of the corresponding nitrogen base using an oxidising agent such as hydrogen peroxide in the presence of an acid such as acetic acid, at an elevated temperature, for example around 70° C. to 80° C., or alternatively by reaction with a peracid such as peracetic acid in a solvent, e.g. dichloromethane, at ambient temperature.

Salts of compounds of formula (1) may be prepared by reaction of a compound of formula (1) with an appropriate base in a suitable solvent or mixture of solvents e.g. an organic solvent such as an ether e.g. diethylether, or an alcohol, e.g. ethanol or an aqueous solvent using conventional procedures. Salts of compounds of formula (1) may be exchanged for other salts by use of conventional ion-exchange chromatography procedures.

Where it is desired to obtain a particular enantiomer of a compound of formula (1) this may be produced from a corresponding mixture of enantiomers using any suitable conventional procedure for resolving enantiomers.

Thus for example diastereomeric derivatives, e.g. salts, may be produced by reaction of a mixture of enantiomers of formula (1) e.g. a racemate, and an appropriate chiral compound, e.g. a chiral base. The diastereomers may then be separated by any convenient means, for example by crystallisation and the desired enantiomer recovered, e.g. by treatment with an acid in the instance where the diastereomer is a salt.

In another resolution process a racemate of formula (1) may be separated using chiral High Performance Liquid Chromatography. Alternatively, if desired a particular enantiomer may be obtained by using an appropriate chiral intermediate in one of the processes described above.

Chromatography, recrystallisation and other conventional separation procedures, may also be used with intermediates or final products where it is desired to obtain a particular geometric isomer of the invention.

The following Examples illustrate the invention. All temperatures are in ° C. The following abbreviations are used:
NMM—N-methylmorpholine;
EtOAc—ethyl acetate;
MeOH—methanol;
BOC—butoxycarbonyl;
DCM—dichloromethane;
AcOH—acetic acid;
DIPEA—diisopropylethylamine;
EtOH—ethanol;
Pyr—pyridine;
DMF—N,N-dimethylformamide;
DMSO—dimethylsulphoxide;
iPr—isopropyl;
Et₂O—diethylether;
Me—methyl;
THF—tetrahydrofuran;
RT—room temperature
LCMS—liquid chromatography-mass spectroscopy

NMR's were obtained at the indicated frequency (quoted as δH or δ³¹P values) The compounds were named with the aid of Beilstein Autonom.

LCMS was performed on a Hewlett Packard 1100 LC/MSD instrument using a Phenomenex Luna 3μ C18(2) 50×4.6 mm column and electrospray ionisation in +ve mode. Compounds were eluted with a mobile phase formed from solution A (0.1% aqueous formic acid) and solution B (0.1% formic acid in acetonitrile) with the following gradient and column conditions. Initial=5% B, 2 min=95% B, 3 min=95% B, 5 min=5% B; column temp 40°; flow rate 0.9 ml min⁻¹; detection UV DAD 210 450 nM.

Intermediate 1

(2R,3R,4S,5R)-2-Benzothiazol-2-yl-5-hydroxymethyl-tetrahydrofuran-3,4-diol Tribenzoate

A mixture of (2S,3R,4S,5R)-3,4-dihydroxy-5-hydroxymethyl-3-methoxy-tetrahydrofuran-2-carbonitrile tribenzoate (490 mg), and 2-aminothiophenol (133 μL) in ethanol (10 ml) under an atmosphere of nitrogen was heated at 50° for 2 h then at reflux for 4 h. The reaction was left to stand at room temperature overnight then adsorbed onto silica and purified by column chromatography (SiO₂; 30% EtOAc/hexane then 2% EtOAc/DCM) to give the title compound (366 mg, 61%) as a while glassy solid. R_{f}0.52 (30% EtOAc in hexane); δH (300 MHz, CDCl₃) 8.10-8.00 (5H, m), 7.95 (2H, d), 7.80 (H, d), 7.60-7.30 (11H, m), 6.10 (H, t), 5.95 (H, t), 5.75 (H, d), 4.95-4.80 (2H, m), 4.65 (H, dd); m/z 581 (M+1).

Intermediate 2

(2R,3S,4R,5R)-2-Hydroxymethyl-5-(5-trifluoromethyl-benzothiazol-2-yl)-tetrahydrofuran-3,4-diol Tribenzoate

Prepared following the procedure for Intermediate 1 from (2S,3R,4S,5R)-3,4-hydroxy-5-hydroxymethyl-3-methoxy-tetrahydrofuran-2-carbonitrile tribenzoate (700 mg), 2-amino-4-trifluoromethyl-benzenethiol hydrochloride (375 mg) and triethylamine (0.23 ml) in ethanol (15 ml), refluxing for 5 h. Purification by column chromatography (SiO₂; 20% EtOAc/hexane then DCM) gave the title compound (400 mg, 42%) as a cream solid. R_{f} 0.40 (DCM); δH (300 MHz, CDCl₃) 8.25 (H, s), 8.05-7.90 (7H, m), 7.65-7.30 (10H, m), 6.10 (H, t), 5.95 (H, t), 5.75 (H, d), 4.95 (H, dd), 4.90-4.80 (H, m), 4.60 (H, dd); m/z 649 (M+1).

Intermediate 3

(2R,3R,4S,5R)-2-Benzothiazol-2-yl-5-hydroxymethyl-tetrahydrofuran-3,4-diol

Intermediate 1 (366 mg) was dissolved in MeOH (5 ml) and DCM (3 ml) under an atmosphere of nitrogen and cooled in an ice bath. Sodium methoxide (136 mg) was added then the reaction allowed to warm to room temperature and stirred for 2 h before quenching with ammonium chloride (270 mg). The reaction was adsorbed onto silica and purified by column chromatography (SiO₂; 30% EtOAc/hexane then 10% MeOH/EtOAc followed by 20% MeOH/EtOAc) to give the title compound (150 mg, 89%) as a white solid. R_{f} 0.70 (10% MeO/EtOAc); δH (300 MHz, DMSO) 8.05 (H, d), 7.95 (H, d), 7.50 (H, t), 7.40 (H, t), 5.45 (H, d), 5.05 (H, d), 5.00 (H, d), 4.85 (H, t), 4.15-4.10 (H, m), 3.95-3.85 (2H, m), 3.65-3.50 (2H, m); m/z 268 (M+1).

Intermediate 4

(2R,3S,4R,5R)-2-Hydroxymethyl-5-(5-trifluoromethyl-benzothiazol-2-yl)-tetrahydrofuran-3,4-diol

Prepared following the procedure for intermediate 3 from Intermediate 2 (400 mg) and sodium methoxide (133 mg) in methanol (5 ml) and DCM (3 ml), quenching with ammonium chloride (265 mg). Purification by column chromatography (SiO₂; 30% EtOAc/hexane followed by 10% MeOH/EtOAc then SiO₂, 5% MeOH/EtOAc) gave the title compound (165 mg, 77%) as a white solid. R_{f} 0.55 (5% MeOH/EtOAc); δH (300 MHz, DMSO) 8.40 (H, s), 8.35 (H, d), 7.75 (H, d), 5.50 (H, d), 5.15 (H, d), 5.05 (H, d), 4.90 (H, t), 4.15-4.10 (H, m), 4.00-3.85 (2H, m), 3.65-3.50 (2H, m); m/z 336 (M+1).

Intermediate 5

2-Amino-5-chloro-benzenethiol

A mixture of 2-amino-6-chloro-benzothiazole (1.5 g), sodium hydroxide (4.5 g) and water (9 ml) was heated at reflux under an atmosphere of nitrogen for 22 h then left to cool. The reaction was poured onto ˜30 ml ice/water, cooled in an ice bath, acidified to pH 3 with 2N HCl then extracted with toluene (3×30 ml). The extracts were washed with brine (40 ml) and dried (MgSO₄). The solvent was removed in vacuo to leave the title compound (833 mg, 64%) as a yellow solid. R_{f} 0.73 (40% hexane/EtOAc); δH (300 MHz, DMSO) 7.20 (H, s), 6.95 (H, d), 6.70 (H, d), 5.35 (H, s).

Intermediate 6

(2R,3R,4S,5R)-3,4-Dihydroxy-5-hydroxymethyl-tetrahydrofuran-2-carbothioic Acid Amide Tribenzoate

Lawesson's reagent (1.96 g) was added to a solution of (2R,3R,4S,5R)-3,4-dihydroxy-5-hydroxymethyl-tetrahydrofuran-2-carboxylic acid amide (4.75 g) in dioxane (50 ml) in an ice-water bath. The mixture was stirred at room temperature for 64 h. The mixture was filtered, concentrated and purified by column chromatography (SiO₂; graded eluent 10% to 25% EtOAc/hexane) to give the title compound as a pale cream solid (3.32 g, 68%). R_{f} 0.65 (50% EtOAc/hexane); δH (300 MHz, CDCl₃) 8.44 (H, broad s) 8.04-7.96 (4H, m), 7.82 (2H, d), 7.60-7.32 (7H, m) 7.28 (2H, t), 5.92 (H, t), 5.64 (H, t), 5.06 (H, d) and 4.70-4.60 (3H, m); m/z 506 (M+1).

Intermediate 7

(2R,3R,4S,5R)-2-(6-Chloro-benzothiazol-2-yl)-5-hydroxymethyl-tetrahydro-furan-3,4-diol Tribenzoate

Prepared following the procedure for Intermediate 1 from (2S,3R,4S,5R)-3,4-hydroxy-5-hydroxymethyl-3-methoxy-tetrahydrofuran-2-carbonitrile tribenzoate (733 mg) and Intermediate 5 (288 mg) in ethanol (15 ml), refluxing for 4.5 h. Purification by column chromatography (SiO₂; 30% EtOAc/hexane) gave the title compound (399 mg, 40%) as a pale yellow solid. R_{f} 0.58 (30% EtOAc/hexane); δH (300 MHz, CDCl₃) 8.05-8.00 (4H, m), 7.95-7.85 (3H, m), 7.75 (H, s), 7.65-7.30 (10H, m), 6.10 (H, t), 5.90 (H, t), 5.70 (H, d), 4.90 (H, dd), 4.85-4.80 (H, m), 4.60 (H, dd); m/z 614 (M+1).

Intermediate 8

(2R,3S,4R,5R)-2-Hydroxymethyl-5-(6-methoxy-benzothiazol-2-yl)-tetrahydro-furan-3,4-diol Tribenzoate

Prepared following the procedure for Intermediate 1 from (2S,3R,4S,5R)-3,4-hydroxy-5-hydroxymethyl-3-methoxy-tetrahydrofuran-2-carbonitrile tribenzoate (1.5 g) and 2-amino-5-methoxy-benzenethiol (0.8 g) in ethanol (25 ml), refluxing for 3 h. Purification by column chromatography (SiO₂; 30% EtOAc/heptane) gave the title compound (1.2 g, 62%) as a pale yellow oil. R_{f} 0.65 (30% EtOAc/heptane); δH (300 MHz, CDCl₃) 8.10-8.05 (4H, m), 7.95-7.85 (3H, m), 7.60-7.50 (3H, m), 7.45-7.30 (6H, m), 7.25 (H, s), 7.05 (H, d), 6.10 (H, t), 5.95 (H, t), 5.75 (H, d), 4.95-4.80 (2H, m), 4.65 (H, dd), 3.85 (3H, s); m/z 610 (M+1).

Intermediate 9

(2R,3S,4R,5R)-2-Hydroxymethyl-5-(4-methoxy-benzothiazol-2-yl)-tetrahydro-furan-3,4-diol Tribenzoate

Prepared following the procedure for Intermediate 1 from (2S,3R,4S,5R)-3,4-hydroxy-5-hydroxymethyl-3-methoxy-tetrahydrofuran-2-carbonitrile tribenzoate (0.39 g) and 2-amino-3-methoxy-benzenethiol (0.34 g) in ethanol (30 ml), refluxing for 18 h. Purification by column chromatography (SiO₂; 20% EtOAc/heptane) gave the title compound (0.28 g, 58%) as a pale yellow oil. R_{f} 0.48 (30% EtOAc/heptane); δH (300 MHz, CDCl₃) 8.10-7.30 (17H, m), 6.80 (H, d), 5.95 (H, t), 5.85 (H, t), 5.80 (H, d), 4.85 (H, dd), 4.70 (H, m), 4.60 (H, dd), 3.95 (3H, s).

Intermediate 10

(2R,3S,4R,5R)-5-(5-Fluorobenzothiazol-2-yl)-2-hydroxymethyl-tetrahydrofuran-3,4-diol Tribenzoate

Intermediate 6 (505 mg), 5-fluoro-2-iodoaniline (237 mg), calcium oxide (56 mg), tris(dibenzylideneacetone)dipalladium(0)-chloroform adduct (50 mg), 1,1′-bis-(diphenylphosphino)ferrocene (110 mg) and dimethylformamide (1 ml) were heated at 60° C. under nitrogen for 2.5 h. The mixture was allowed to cool, and purification by column chromatography (SiO₂; graded eluent 10% to 20% EtOAc/hexane) gave the title compound as a colourless gum (205 mg, 31%). R_{f} 0.55 (30% EtOAc/hexane); δH (300 MHz, CDCl₃) 8.04 (4H, d), 7.94 (2H, d), 7.74 (H, dd), 7.66 (H, dd), 7.64-7.32 (9H, m) 7.16 (H, t of d), 6.08 (H, t), 5.92 (H, t), 5.74 (H, d), 4.92 (H, dd), 4.84 (H, ddd) and 4.64 (H, dd); m/z 598 (M+1).

Intermediate 11

(2R,3S,4R,5R)-5-(6-Chloro-4-fluorobenzothiazol-2-yl-2-hydroxymethyl-tetrahydrofuran-3,4-diol Tribenzoate

Prepared following the procedure for Intermediate 10 from Intermediate 6 (606 mg), 2-chloro-2-fluoro-6-iodoaniline (326 mg), calcium oxide (67 mg), tris(dibenzylideneacetone)dipalladium(0)-chloroform adduct (62 mg), 1,1′-bis-(diphenylphosphino)ferrocene (133 mg) and dimethylformamide (1.2 ml), heating at 60° C. for 2 h. Purification by column chromatography (SiO₂; graded eluent 10% to 20% EtOAc/hexane) gave the title compound as a colourless gum (146 mg, 19%). Rₜ 0.5 (30% EtOAc/hexane); δH (300 MHz, CDCl₃) 8.06 (2H, d), 8.00 (2H, d), 7.94 (2H, d), 7.64-7.50 (4H, m), 7.48-7.30 (6H, m), 7.20 (H, dd), 6.06 (H, t), 5.94 (H, t), 5.76 (H, d), 4.94 (H, dd), 4.82 (H, ddd) and 4.62 (H, dd); m/z 632 (M+1).

Intermediate 12

(2R,3S,4R,5S)-2-Hydroxymethyl-5-(1-methyl-1H-benzoimidazol-2-yl)-tetrahydro-furan-3,4-diol Tribenzoate

Oxalyl chloride (0.5 g) was added to a solution of (2R,3R,4S,5R)-3,4-dihydroxy-5-hydroxymethyl-tetrahydro-furan-2-carboxylic acid tribenzoate (0.35 g) in DCM (40 ml). A drop of DMF was added and the mixture stirred until all effervesence had ceased. Phenylenediamine (0.22 g) was added and stirring continued overnight. The solvent was removed in vacuo, and the residue dissolved in toluene (20 ml). Phosphorous oxychloride (0.1 g) and triethylamine (0.07 g) were added, and the mixture heated to reflux overnight. After cooling to rt it was cautiously quenched with a saturated aqueous solution of sodium bicarbonate. The organics were separated, dried (MgSO₄), evaporated and chromatographed (SiO₂; 30% EtOAc/hexane). The resulting residue was dissolved in THF (5 ml) and sodium hydride (0.02 g) was added. The mixture was stirred at rt for 10 min before addition of iodomethane (0.07 g) and then overnight. The mixture was partitioned between water (20 ml) and dichloromethane (20 ml). The organics were dried (MgSO₄), evaporated and purified by chromatography (SiO₂; 4% methanol-DCM) to give the title compound as a light brown solid (180 mg, 19%). Rₜ 0.75 (4% methanol-DCM); δH (300 MHz, CDCl₃) 8.10 (2H, d), 8.00 (H, d), 7.85 (2H, t), 7.50-7.40 (4H, m), 7.40-7.25 (8H, m), 5.90 (H, t), 5.80 (H, t), 4.90 (H, d), 4.70-4.60 (2H, m), 4.55-4.45 (H, m), 3.40 (3H, s); m/z 577 (M+1).

Intermediate 13

(2R,3S,4R,5S)-2-Hydroxymethyl-5-(1-methyl-1H-benzoimidazol-2-yl)-tetrahydro-furan-3,4-diol

Prepared following the procedure for Intermediate 3 from Intermediate 12 (180 mg) and sodium methoxide (60 mg) in methanol (3 ml) and EtOAc (12 ml). Trituration with hexane gave the title compound (72 mg, 87%) as a pale brown solid. R_{f} 0.41 (10% methanol-DCM; δH (300 MHz, DMSO) 7.80 (H, d), 7.65 (H, d), 7.55-7.45 (2H, m), 5.45 (H, d), 4.85 (2H, br s), 4.60 (H, br s), 4.55 (H, br s), 4.30 (H, m), 4.05 (H, dd), 4.05 (3H, s), 3.80 (H, dd); m/z 265 (M+1).

Intermediate 14

(2R,3R,4S,5R)-2-(6-Chloro-benzothiazol-2-yl)-5-hydroxymethyl-tetrahydro-furan-3,4-diol

Prepared following the procedure for Intermediate 3 from Intermediate 7 (399 mg) and sodium methoxide (140 mg) in methanol (5 ml) and DCM (3 ml), quenching with ammonium chloride (280 mg). Purification by column chromatography (SiO₂; 30% EtOAc/hexane then EtOAc followed by 10% MeOH/EtOAc) gave the title compound (155 mg, 79%) as a white solid. R_{f} 0.40 (EtOAc); δH (300 MHz, DMSO) 8.30 (H, d), 8.00 (H, d), 7.55 (H, dd), 5.50 (H, d), 5.15 (H, d), 5.05 (H, d), 4.90 (H, t), 4.15-4.10 (H, m), 4.00-3.90 (2H, m), 3.70-3.55 (2H, m); m/z 302 (M+1).

Intermediate 15

(2R,3S,4R,5R)-2-Hydroxymethyl-5-(6-methoxy-benzothiazol-2-yl)-tetrahydro-furan-3,4-diol

Prepared following the procedure for Intermediate 3 from Intermediate 8 (1.2 g) and sodium methoxide (420 mg) in methanol (10 ml) and ethyl acetate (10 ml), quenching with ammonium chloride (0.5 g). Purification by column chromatography (SiO₂;50:45:5 heptane/EtOAc/MeOH) gave the title compound (250 mg, 43%) as a white solid. R_{f} 0.12 (50:45:5 heptane/EtOAc/MeOH); δH (300 MHz, CDCl₃) 7.85(H, d), 7.30 (H, d), 7.10 (H, dd), 5.25 (H, d), 4.50-4.40 (2H, m), 4.20-4.25(H, m), 3.90 (3H, s), 3.25 (H, d), 2.75(H, d); m/z 298 (M+1).

Intermediate 16

(2R,3S,4R,5R)-2-Hydroxymethyl-5-(4-methoxy-benzothiazol-2-yl)-tetrahydro-furan-3,4-diol

Prepared following the procedure for Intermediate 3 from Intermediate 9 (0.28 g) and sodium methoxide (0.10 g) in methanol (10 ml) and DCM (10 ml), quenching with ammonium chloride (0.20 g). Purification by column chromatography (SiO₂; 5% MeOH/DCM) to give the title compound (0.118 g, 88%) as a white solid. R_{f} 0.28 (5% MeOH/DCM); ); δH (300 MHz, DMSO) 7.4 (H, d), 7.1 (H, t), 6.8 (H, d), 5.2 (H, d), 4.8 (H, d), 4.75 (H, d), 4.6 (H, t), 3.9 (H, t), 3.7-3.6 (2H, m), 3.7 (3H, s), 3.4-3.3 (2H, m); m/z 298 (M+1).

Intermediate 17

(2R,3R,4S,5R)-2-(5-Fluorobenzothiazol-2-yl)-5-hydroxymethyl-tetrahydrofuran-3,4-diol

Prepared following the procedure for Intermediate 3 from Intermediate 10 (280 mg) and sodium methoxide (101 mg) in methanol (5 ml) and DCM (5 ml), quenching with ammonium chloride (200 mg). Purification by column chromatography (SiO₂; 20% EtOAc/hexane followed by EtOAc) gave the title compound (66 mg, 49%) as a beige solid. R_{f} 0.45 (EtOAc); SH (300 MHz, DMSO) 8.18 (H, dd), 7.86 (H, dd), 7.38 (H, t of d), 5.52 (H, d), 5.16 (H, d), 5.06 (H. d), 4.92 (H, t), 4.14 (H, q),.4.00-3.90 (2H, m) and 3.68-3.54 (2H, m); m/z 286 (M+1).

Intermediate 18

(2R,3R,4S,5R)-2-(6-Chloro-4-fluoro-benzothiazol-2-yl)-5-hydroxymethyl-tetrahydrofuran-3,4-diol

Prepared following the procedure for Intermediate 3 from Intermediate 11 (140 mg) and sodium methoxide (48 mg) in methanol (5 ml) and DCM (5 ml), quenching with ammonium chloride (100 mg). Purification by column chromatography (SiO₂; 20% EtOAc/hexane followed by EtOAc) gave the title compound (60 mg, 80%) as a beige solid. R_{f} 0.45 (EtOAc); δH (300 MHz, DMSO) 8.04 (H, d), 7.48 (H, dd), 5.44 (H, d), 5.02 (H, d), 4.92 (H, d), 4.78 (H, t), 4.00 (H, q), 3.88-3.76 (2H, m) and 3.54-3.38 (2H, m); m/z 320 (M+1).

Intermediate 19

(2R,3S,4R,5R)-2-Hydroxymethyl-5-(5-methyl-benzooxazol-2-yl)-tetrahydro-furan-3,4-diol

(2R,3R,4S,5R)-3,4-Dihydroxy-5-hydroxymethyl-tetrahydro-furan-2-carboximidic acid methyl ester (320 mg) was dissolved in THF(10 ml). Hydrogen chloride (5 ml of a 1.0N solution in ether) was added and the mixture stirred for 15 min. The resulting solid was removed by filtration, washed with THF (50 ml) and dissolved in methanol (20 ml). 2-Amino-p-cresol (206 mg) was added, and the mixture heated to reflux for 4 h. The reaction mixture was adsorbed onto silica and purified by column chromatography (SiO₂; EtOAc followed by 5% MeOH/EtOAc) to give the title compound (150 mg, 34%) as a white solid. R_{f} 0.36 (EtOAc); δH (300 MHz, DMSO) 7.65 (H, d), 7.60 (H, s), 7.30 (H, d), 5.45 (H, d), 5.20 (H, d), 4.80 (2H, d overlapping with t), 4.40 (H, t), 4.10 (H, t), 4.00-3.90 (H, m), 3.60-3.40 (2H, m), 2.45 (3H, s); m/z 266 (M+1).

### EXAMPLE 1

### (2R,3S,4R,5R)-3,4-Dihydroxy-5-benzothiazol-2-yl-tetrahydrofuran-2-ylmethyl Triphosphate tris-ammonium Salt

Trimethylphosphate (2.2 ml) was added to a mixture of Intermediate 3 (150 mg) and proton sponge (180 mg) at room temperature under nitrogen. The mixture was stirred at room temperature until all the solid had dissolved and was then cooled in an ice bath. Phosphorous oxychloride (0.058 ml) was added dropwise and the mixture stirred for 2 h before simultaneous addition of tributylamine (0.56 ml) and a 0.5M solution of tributylammonium pyrophosphate in DMF (5.6 ml). The mixture was stirred for a further 2.5 min then quenched by addition of aqueous ammonium bicarbonate (140 mg in 14 ml of water) and stirred for 2 h. The volatile solvents were evaporated in vacuo and the residue purified by reverse phase ion-pair chromatography on a Luna C18 column eluting with an ammonium acetate buffer. Fractions containing the desired triphosphate were extracted onto an ion exchange SPE cartridge which was eluted with 10% aqueous ammonia to give, after evaporation of the solvent in vacuo, the title compound as a white gum (23 mg). δH (400 MHz, D₂O) 8.10 (H, d), 8.00 (H, d), 7.60 (H, t), 7.50 (H, t), 5.30 (H, d) 4.50-4.35 (3H, m), 4.30-4.25 (2H, m); δ³¹P (162 MHz, D₂O) −7.0 (d, J_{P-P}=19 Hz), −10.1 (d, J_{P-P}=20 Hz), −21.3 (t, J_{P-P}=20 Hz); m/z 506 (M−1).

### EXAMPLE 2

### (2R,3S,4R,5R)-3,4-Dihydroxy-5-(5-trifluoromethyl-benzothiazol-2-yl)-tetrahydrofuran-2-ylmethyl Triphosphate tris-ammonium Salt

Prepared following the procedure for Example 1 from Intermediate 4 (163 mg), proton sponge (156 mg) and phosphorous oxychloride (0.050 ml) in trimethyl phosphate (1.9 ml), then tributylamine (0.49 ml) and 0.5M tributylammonium pyrophosphate in DMF (4.9 ml). The reaction was quenched with aqueous ammonium bicarbonate (122 mg in 12 ml of water) and stirred for 1.5 h. Purification as for Example 1 gave the title compound as a white solid (30 mg). δH (400 MHz, D₂O) 8.35 (H, s), 8.25 (H, d), 7.80 (H, d), 5.30 (H, d), 4.55-4.40 (3H, m), 4.30-4.25 (2H, m); δ³¹P (162 MHz, D₂O) −6.0 (d, J_{P-P}=20 Hz), −9.9 (d, J_{P-P}=20 Hz), −21.1 (t, J_{P-P}=20 Hz); m/z 574 (M−1).

### EXAMPLE 3

### (2R,3S,4R,5R)-3,4-Dihydroxy-5-(6-chlorobenzothiazol-2-yl)-tetrahydrofuran-2-ylmethyl Triphosphate tris-ammonium Salt

Prepared following the procedure for Example 1 from Intermediate 14 (155 mg), proton sponge (165 mg) and phosphorous oxychloride (0.053 ml) in trimethyl phosphate (2.0 ml), then tributylamine (0.51 ml) and 0.5M tributylammonium pyrophosphate in DMF (5.1 ml). The reaction was quenched with aqueous ammonium bicarbonate (129 mg in 13 ml of water) and stirred for 2 h. Purification as for Example 1 gave the title compound as a white solid (30 mg). δH (400 MHz, D₂O) 8.15 (H, s), 7.95 (H, d), 7.60 (H, d), 5.25 (H, d), 4.50-4.45 (2H, m), 4.40-4.35 (H, m), 4.30-4.25 (2H, m); δ³¹P (162 MHz, D₂O) −5.8-−6.1 (m), −10.0 (d, J_{P-P}=20 Hz), −21.2 (t, J_{P-P}=20 Hz); m/z 540 (M−1).

### EXAMPLE 4

### (2R,3S,4R,5R)-3,4-Dihydroxy-5-(6-methoxybenzothiazol-2-yl)-tetrahydrofuran-2-ylmethyl Triphosphate tris-Ammonium Salt

Prepared following the procedure for Example 1 from Intermediate 15 (53 mg), proton sponge (57 mg) and phosphorous oxychloride (0.018 ml) in trimethyl phosphate (0.8 ml), then tributylamine (0.21 ml) and 0.5M tributylammonium pyrophosphate in DMF (1.79 ml). The reaction was quenched with aqueous ammonium bicarbonate (44 mg in 4.5 ml of water). Purification as for Example 1 gave the title compound as a colourless gum (30 mg). δH (400 MHz, D₂O) 7.90 (H, d), 7.65 (H, s), 7.20 (H, d), 5.25 (H, d), 4.50-4.40 (2H, m), 4.40-4.35 (H, m), 4.30-4.25 (2H, m); δ³¹P (162 MHz, D₂O) −7.8 (d, J_{P-P}=19 Hz), −10.1 (d, J_{P-P}=20 Hz), −21.5 (t, J_{P-P}=20 Hz); m/z 536 (M−1).

### EXAMPLE 5

### (2R,3S,4R,5R)-3,4-Dihydroxy-5-(4-methoxy-benzothiazol-2-yl)-tetrahydrofuran-2-ylmethyl Triphosphate tris-Ammonium Salt

Prepared following the procedure for Example 1 from Intermediate 16 (118 mg), proton sponge (127.5 mg) and phosphorous oxychloride (0.041 ml) in trimethyl phosphate (1.6 ml), then tributylamine (0.40 ml) and 0.5M tributylammonium pyrophosphate in DMF (4.0 ml). The reaction was quenched with aqueous ammonium bicarbonate (99 mg in 10 ml of water). Purification as for Example 1 gave the title compound as a white solid (14.9 mg). δH (400 MHz, D₂O) 7.70 (H, d), 7.45 (H, t), 7.15 (H, d), 5.25 (H, d), 4.5-4.4 (2H, m), 4.4-4.35 (H, m), 4.3-4.2 (2H, m), 4.0 (3H, s); δ³¹P (162 MHz, D₂O) −5.2 (d, J_{P-P}=21 Hz), −-9.9 (d, J_{P-P}=20 Hz), −20.8 (t, J_{P-P}=20 Hz) m, z 536 (M−1).

### EXAMPLE 6

### (2R,3S,4R,5R)-3,4-Dihydroxy-5-(5-fluorobenzothiazol-2-yl)-tetrahydrofuran-2-ylmethyl Triphosphate tris-Triethylammonium Salt

Prepared following the procedure for Example 1 from Intermediate 17 (62 mg), proton sponge (70 mg) and phosphorous oxychloride (0.022 ml) in trimethyl phosphate (1 ml), then tributylamine (0.22 ml) and 0.5M tributylammonium pyrophosphate in DMF (2.2 ml). The reaction was quenched with aqueous ammonium bicarbonate (54 mg in 5 ml of water) and stirred for 0.5 h. Purification as for Example 1, but eluting the SPE cartridge with triethylamine instead of ammonia, gave the title compound as a white solid (30 mg). δH (400 MHz, D₂O) 8.08 (H, dd), 7.74 (H, dd), 7.34 (H, t of d), 5.28 (H, d), 4.50-4.36 (3H, m), 4.32-4.24 (2H, m), 3.20 (18H, q) and 1.26 (27H, t); δ³¹P (162 MHz, D₂O) −8.1 (d, J_{P-P}=19.5 Hz), −10.1 (d, J_{P-P}=19.5 Hz) and −21.5 (t, J_{P-P}=19.5 Hz); m/z 524 (M−1).

### EXAMPLE 7

### (2R,3S,4R,5R)-5-(6-Chloro-4-fluorobenzothiazol-2-yl)-3,4-dihydroxy-tetrahydrofuran-2-ylmethyl Triphosphate tris-Triethylammonium Salt

Prepared following the procedure for Example 1 from Intermediate 18 (59 mg), proton sponge (59 mg) and phosphorous oxychloride (0.019 ml) in trimethyl phosphate (1 ml), then tributylamine (0.18 ml) and 0.5M tributylammonium pyrophosphate in DMF (1.8 ml). The reaction was quenched with aqueous ammonium bicarbonate (46 mg in 5 ml of water) and stirred for 0.5 h. Purification as for Example 1, but eluting the SPE cartridge with triethylamine instead of ammonia, gave the title compound as a white solid (14 mg). δH (400 MHz, D₂O) 7.94 (H, s), 7.40 (H, d), 5.28 (H, d), 4.50-4.38 (3H, m), 4.30-4.24 (2H, m), 3.20 (18H, q) and 1.28 (27H, t); δ³¹P (162 MHz, D₂O) −7.5 (d, J_{P-P}=20 Hz), −10.1 (d, J_{P-P}=20 Hz) and −21.5 (t, J_{P-P}=20 Hz); m/z 558 (M−1).

### EXAMPLE 8

### (2R,3S,4R,5S)-2-Hydroxymethyl-5-(1-methyl-1H-benzoimidazol-2-yl)-tetrahydro-furan-3,4-diol Triphosphate tris-Ammonium Salt

Prepared following the procedure for Example 1 from Intermediate 13 (72 mg), proton sponge (87 mg) and phosphorous oxychloride (0.027 ml) in trimethyl phosphate (1.2 ml), then tributylamine (0.32 ml) and 0.5M tributylammonium pyrophosphate in DMF (2.73 ml). The reaction was quenched with aqueous ammonium bicarbonate (68 mg in 6.8 ml of water) and stirred for 0.5 h. Purification as for Example 1 gave the title compound as a white solid (9 mg). δH (400 MHz, D₂O) 7.80 (H, d), 7.70 (H, d), 7.50-7.40 (2H, m), 5.25 (H, d), 4.70 (H, t), 4.50 (H, t), 4.40 (H, d), 4.20 (2H, m), 4.00 (3H, s); δ³¹P (162 MHz, D₂O) −7.3 (m), −9.7 (d, J_{P-P}=19 Hz) and −20.8 (t, J_{P-P}=19 Hz); m/z 503 (M−1).

### EXAMPLE 9

### (2R,3S,4R,5R)-2-Hydroxymethyl-5-(5-methyl-benzooxazol-2-yl)-tetrahydro-furan-3,4-diol Triphosphate tris-Ammonium Salt

Prepared following the procedure for Example 1 from Intermediate 19 (145 mg), proton sponge (156 mg) and phosphorous oxychloride (0.056 ml) in trimethyl phosphate (2.2 ml), then tributylamine (0.55 ml) and 0.5M tributylammonium pyrophosphate in DMF (5.5 ml). The reaction was quenched with aqueous ammonium bicarbonate (137 mg in 14 ml of water) and stirred for 1.5 h. Purification as for Example 1 gave the title compound as a white gum (20 mg). δH (400 MHz, D₂O) 7.60 (2H, m), 7.35 (H, d), 5.25 (H, d), 4.70 (H, t), 4.50 (H, t), 4.40 (H, m), 4.20 (2H, m), 2.45 (3H, s); δ³¹P (162 MHz, D₂O) −4.8 (d, J_{P-P}=20 Hz), −9.6 (d, J_{P-P}=19 Hz), −20.3 (m); m/z 504 (M−1).

The following assays can be used to demonstrate the potency and selectivity of the compounds according to the invention.

P2Y2 Assay

The P2Y2 clone was isolated from placental cDNA by PCR, using specific primers, inserted between the Not1 and EcoR1 sites in the multi-cloning site of the pIRESpuro vector (Clontech). The vector was stably transfected into a human astrocytoma cell-line, 1321 N1, and raised under puromycin selection. The cells were maintained in Dulbecco's MEM growth medium, containing 10% fcs, 2 mM glutamine, 1% non-essential amino acids, 2 μg/ml puromycin, at 37° C. with 5% CO₂ and grown to sub-confluence, before removing with trypsin and re-seeding. Prior to assay, cells were seeded at 1×10⁴ cells/well in 100 μl of growth medium in a 96-well black walled, clear bottomed tissue culture plate and incubated at 37° C. overnight.

The culture medium was gently removed from the wells and replaced with wash buffer (Hank's Balanced Salts Solution with 0.2% BSA and 20 mM HEPES pH 7.2) containing 2 μM Fluo-4 and 0.02% pluronic acid. The plate was incubated at 37° C. for 1 hour, then gently washed twice and 100 μl wash buffer added per well.

The calcium response assay was performed in a FLIPR™ (Molecular Devices). The compound of the invention was dissolved in DMSO and then diluted in wash buffer to give a DMSO concentration of 0.3% (reduced to 0.1% when added to the assay plate in the FLIPR™). The compound was added to the assay plate after a 10 second baseline. After a further 3 minutes a UTP stimulus was added. The response of the compound was compared to that of UTP.

Human P2Y4 receptors were cloned from genomic DNA by PCR, whilst P2Y6 receptors were isolated from a human peripheral blood mononuclear cell cDNA library. These receptors were stably expressed in 1321N1 cells and assayed as described above for P2Y2 receptors.

In the above assays the preferred compound of the invention generally has EC₅₀ values in the P2Y2, P2Y6 and/or P2Y4 assays of 10 μM and below.

1. A compound useful for modulating P2Y receptor activity of formula (1): 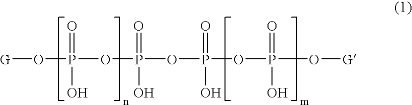
wherein:
G is a hydrogen atom or an optionally substituted aliphatic, heteroaliphatic, cycloaliphatic, heterocycloaliphatic, polycycloaliphatic, heteropolycyclo-aliphatic, aromatic or heteroaromatic group or a group of formula: 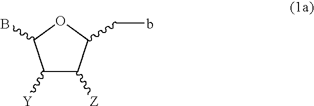
Y and Z are each independently a hydrogen or halogen atom or a hydroxyl (—OH), alkoxy, azido (—N₃), amino (—NH₂), alkylamino or dialkylamino group;
b represents the point of attachment to the remainder of the compound of formula (1);
B is an optionally substituted carbon-linked bicyclic heteroaromatic group;
G′ is a group of formula: 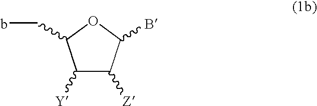
B′ is an optionally substituted carbon-linked bicyclic heteroaromatic group;
Z′ and Y′ are each independently a hydrogen or halogen atom or a hydroxyl (—OH), alkoxy, azido (—N₃), amino (—NH₂), alkylamino or dialkylamino group;
b represents the point of attachment to the remainder of the compound of formula (1);
n is zero, or the integer 1 or 2;
m is zero or the integer 1 or 2;
and the salts, solvates, hydrates and N-oxides thereof.

2. A compound of formula (1c): 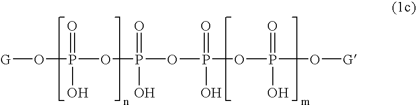
wherein:
G is a hydrogen atom or an optionally substituted aliphatic, heteroaliphatic, cycloaliphatic, heterocycloaliphatic, polycycloaliphatic, heteropolycycloaliphatic, aromatic or heteroaromatic group or a group of formula: 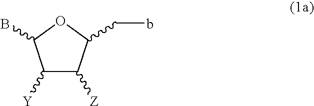
Y and Z are each independently a hydrogen or halogen atom or a hydroxyl (—OH), alkoxy, azido (—N₃), amino (—NH₂), alkylamino or dialkylamino group;
b represents the point of attachment to the remainder of the compound of formula (1);
B is an optionally substituted carbon-linked bicyclic heteroaromatic group;
G′ is a group of formula: 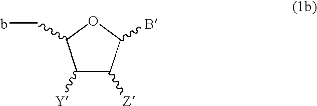
B′ is an optionally substituted carbon-linked bicyclic heteroaromatic group;
Z′ and Y′ are each independently a hydrogen or halogen atom or a hydroxyl (—OH), alkoxy, azido (—N₃), amino (—NH₂), alkylamino or dialkylamino group;
b represents the point of attachment to the remainder of the compound of formula (1);
n is zero, or the integer 1 or 2;
m is zero or the integer 1 or 2;
provided that:
1) when n and m are each zero, G is a hydrogen atom and G′ is a group of formula (1b) in which Y′ and Z′ are each a hydroxyl (—OH) group, then B′ is other than a 7H-pyrazolo[4,3-d]pyrimidine-7-one-3-yl, 7-amino-1H-pyrazolo[4,3-d]pyrimidin-3-yl, 4H-pyrrolo[3,2-d]pyrimidin-4-one-7-yl or 4-amino-4H-pyrrolo[3,2-d]pyrimidin-7-yl group;
2) when one of n and m is the integer 1 and the other is zero, G is a hydrogen atom and G′ is a group of formula (1b) in which Y′ and Z′ are each a hydroxyl (—OH) group, then B′ is other than a 7H-pyrazolo[4,3-d]pyrimidine-7-one-3-yl, 5-amino-7H-pyrazolo[4,3-d]pyrimidine-7-one-3-yl, 7-amino-1H-pyrazolo[4,3-d]pyrimidin-3-yl, 2-amino-4H-pyrrolo[3,2-d]pyrimidin-4-one-7-yl, 4H-pyrrolo[3,2-d]pyrimidin-4-one-7-yl, 2-amino-4H-pyrrolo[3,2-d]pyrimidin-4-one-7-yl, 4-aminothieno[3,2-d]pyrimidin-7-yl, 4-amino-4H-pyrrolo[3,2-d]pyrimidin-7-yl or 4-amino-5H-pyrrolo[3,2-d]pyrimidin-7-yl group;
3) when n and m are each zero, G′ is a group of formula (1b) in which Y′ and Z′ are each a hydroxyl (—OH) group, B′ is a 7-amino-1H-pyrazolo[4,3-d]pyrimidin-3-yl group and G is a group of formula (1a) in which Y′ and Z′ are each a hydroxyl (—OH) group, then B is other than a 7-amino-1H-pyrazolo[4,3-d]pyrimidin-3-yl group;
and the salts, solvates, hydrates and N-oxides thereof.

3. A compound according to claim 1 in which G′ has the formula (1b) in which the furanose sugar has the β-configuration.

4. A compound according to claim 3 in which G′ has the β-D-configuration.

5. A compound according to claim 4 in which G′ has the β-D-ribofuranose configuration.

6. A compound according to claim 1 of formula (2a): 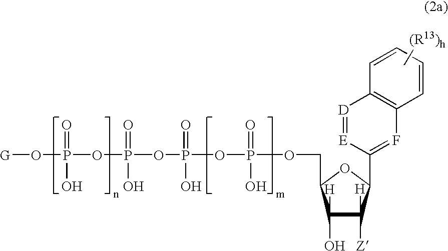
wherein:
D, E and F are each a carbon or nitrogen atom provided that no more than two of D, E and F are a nitrogen atom;
Z′ is a hydroxyl (—OH), amino (—NH₂) or azido (—N₃) group;
the ribose sugar is of a natural β-D configuration as shown;
h is zero or the integer 1, 2, 3 or 4;
R¹³ is an optional substituent that may be on any available carbon atom of the heterocyclic ring B′;
and the salts, solvates, hydrates and N-oxides thereof.

7. A compound according to claim 6 in which D and E are each a carbon atom and F is a nitrogen atom.

8. A compound according to claim 6 in which D and F are each a carbon atom and E is a nitrogen atom.

9. A compound according to claim 6 in which E and F are each a carbon atom and D is a nitrogen atom.

10. A compound according to claim 6 in which E and F are each a nitrogen atom and D is a carbon atom.

11. A compound according to claim 1 of formula (2b): 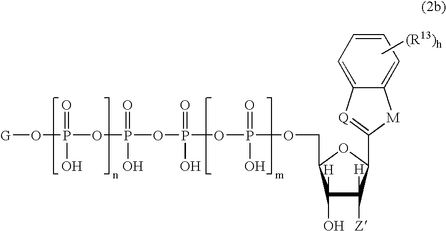
wherein:
Q is a N atom or a CH or C(R ¹³) group;
M is an oxygen or sulphur atom or an NH or N(R¹³) group;
Z′ is a hydroxyl (—OH), amino (—NH₂) or azido (—N₃) group;
the ribose sugar is of a natural β-D configuration as shown;
h is zero or the integer 1, 2, 3 or 4;
R¹³ is an optional substituent which may be on any available carbon or nitrogen atom of the heterocyclic ring B′;
and the salts, solvates, hydrates and N-oxides thereof.

12. A compound according to claim 11 in which M is an oxygen atom and Q is a CH or C(R¹³) group.

13. A compound according to claim 11 in which M is a sulphur atom and Q is a CH or C(R¹³) group.

14. A compound according to claim 11 in which M is an NH or N(R¹³) group and Q is a CH or C(R¹³) group.

15. A compound according to claim 11 in which Q is a N atom and M is an oxygen or sulphur or NH or N(R¹³) group.

16. A compound according to claim 11 in which R¹³ is a —CH₃ group.

17. A compound according to claim 1 in which G is a hydrogen atom or an optionally substituted aliphatic, heteroaliphatic, cycloaliphatic, polycycloaliphatic, aromatic or heteroaromatic group or a group of formula (1a).

18. A compound according to claim 1 in which G is a group of formula (2c): 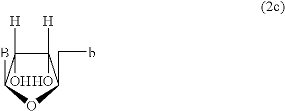
in which b indicates the point of attachment to the remainder of the compound of formula (1).

19. A compound according to claim 18 in which B is a group of formula (2d): 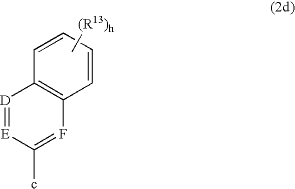
wherein:
c represents the point of attachment to the molecule of formula (2c);
D, E and F are each a carbon or nitrogen atom provided that no more than two of D, E and F are a nitrogen atom;
Z′ is a hydroxyl (—OH), amino (—NH₂) or azido (—N₃) group;
the ribose sugar is of a natural β-D configuration as shown;
h is zero or the integer 1, 2, 3 or 4;
R¹³ is an optional substituent that may be on any available carbon atom of the heterocyclic ring B′.

20. A compound according to claim 18 in which B is a group of formula (2e): 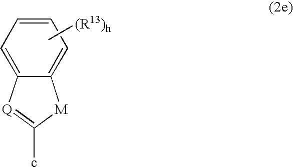
wherein:
c represents the point of attachment to the molecule of formula (2c);
Q is a N atom or a CH or C(R¹³) group;
M is an oxygen or sulphur atom or an NH or N(R¹³) group;
Z′ is a hydroxyl (—OH), amino (—NH₂) or azido (—N₃) group;
the ribose sugar is of a natural β-D configuration as shown;
h is zero or the integer 1, 2, 3 or 4;
R¹³ is an optional substituent which may be on any available carbon or nitrogen atom of the heterocyclic ring B′.

21. A compound according to claim 1 in which G is a group of formula (1a) and G′ is a group of formula (1b) in which B and B′ are each a group of formula (2d): 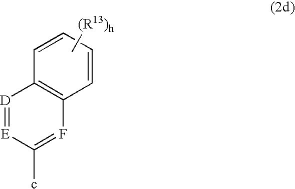
wherein:
c represents the point of attachment to the molecules of formulas (1a) and (1b);
D, E and F are each a carbon or nitrogen atom provided that no more than two of D, E and F are a nitrogen atom;
Z′ is a hydroxyl (—OH), amino (—NH₂) or azido (—N₃) group;
the ribose sugars are of a natural β-D configuration ;
h is zero or the integer 1, 2, 3 or 4;
R¹³ is an optional substituent that may be on any available carbon atom of the heterocyclic ring of formula (2d);
or B and B′ are each a group of formula (2e): 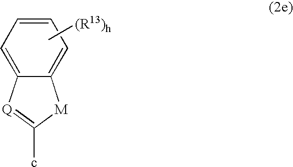
wherein:
c represents the point of attachment to the molecule of formula (2c);
Q is a N atom or a CH or C(R¹³) group;
M is an oxygen or sulphur atom or an NH or N(R¹³) group;
Z′ is a hydroxyl (—OH), amino (—NH₂) or azido (—N₃) group;
the ribose sugars are of a natural β-D configuration;
h is zero or the integer 1, 2, 3 or 4;
R¹³ is an optional substituent which may be on any available carbon or nitrogen atom of the heterocyclic ring B′.

22. A compound according to claim 6 in which G is a group of formula (2c): 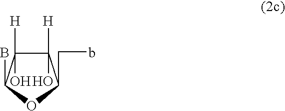
wherein:
b indicates the point of attachment to the remainder of the compound of formula (2a); and
B is a group of formula (2d): 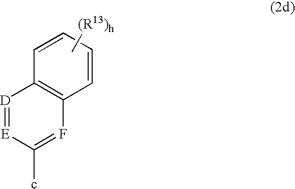
wherein:
c represents the point of attachment to the molecule of formula (2c);
D, E and F are each a carbon or nitrogen atom provided that no more than two of D, E and F are a nitrogen atom;
Z′ is a hydroxyl (—OH), amino (—NH₂) or azido (—N₃) group;
the ribose sugar is of natural β-D configuration as shown;
h is zero or the integer 1, 2, 3 or 4;
R¹³ is an optional substituent that may be on any available carbon atom of the heterocyclic ring of formula (2d).

23. A compound according to claim 6 in which G is a group of formula (2c): 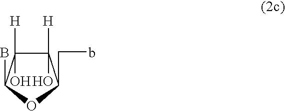
wherein:
b indicates the point of attachment to the remainder of the compound of formula (2a); and
B is a group of formula (2e): 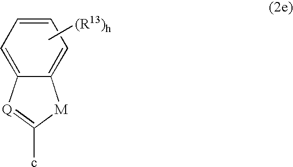
wherein:
c represents the point of attachment to the molecule of formula (2c);
Q is a N atom or a CH or C(R¹³) group;
M is an oxygen or sulphur atom or an NH or N(R¹³) group;
Z′ is a hydroxyl (—OH), amino (—NH²) or azido (—N₃) group;
the ribose sugar is of natural β-D configuration as shown;
h is zero or the integer 1, 2, 3 or 4;
R¹³ is an optional substituent which may be on any available carbon or nitrogen atom of the heterocyclic ring of formula (2e).

24. A compound according to claim 22 in which B and B′ are identical.

25. A compound according claim 1 in which G is a hydrogen atom.

26. A compound according to claim 25 in which m is the integer 1 and n is zero.

27. A compound which is
(2R,3S,4R,5R)-3,4-Dihydroxy-5-benzothiazol-2-yl-tetrahydrofuran-2-ylmethyl triphosphate tris-ammonium salt;
(2R,3S,4R,5R)-3,4-Dihydroxy-5-(5-trifluoromethyl-benzothiazol-2-yl)-tetrahydrofuran-2-ylmethyl triphosphate tris-ammonium salt;
and the free acid, other pharmaceutically acceptable salts, solvates, hydrates and N-oxides thereof.

28. A compound which is:
(2R,3S,4R,5R)-3,4-Dihydroxy-5-(6-chlorobenzothiazol-2-yl)-tetrahydrofuran-2-ylmethyl triphosphate tris-ammonium salt;
(2R,3 S,4R,5R)-3,4-Dihydroxy-5-(5-fluorobenzothiazol-2-yl)-tetrahydrofuran-2-ylmethyl triphosphate tris-triethylammonium salt;
(2R,3 S,4R,5R)-5-(6-Chloro-4-fluorobenzothiazol-2-yl)-3,4-dihydroxy-tetrahydrofuran-2-ylmethyl triphosphate tris-triethylammonium salt;
and the free acid, other pharmaceutically acceptable salts, solvates, hydrates and N-oxides thereof.

29. A pharmaceutical composition comprising a compound according to claim 1 together with one or more pharmaceutically acceptable carriers, excipients or diluents.

30. (canceled)

31. A method for the treatment of a lung disorder involving inadequate functioning of mucociliary clearance mechanisms comprising administering to a mammal suffering from such a disorder a therapeutically effective amount of a compound of claim 1.

32. The method of claim 31 wherein the disorder is chronic bronchitis, primary ciliary dyskinesia, cystic fibrosis, sinusitis, otitis media, post-operative mucous retention, nasolacrimal duct obstructions, or female infertility or irritation.
